(19) United States
(12) Patent Application Publication
(10) Pub. No.: US 2019/0281871 A1
(43) Pub. Date: Sep. 19, 2019

(54) CHICORY PRODUCTS CONTAINING CANNABINOIDS

(71) Applicant: Full Spectrum Laboratories Ltd, Dublin (IL)

(72) Inventors: Richard C. Peet, Washington, DC (US); Malcolm J. Kavarana, Fairfax, VA (US)

(21) Appl. No.: 15/571,646

(22) PCT Filed: Oct. 4, 2017
(86) PCT No.: PCT/EP2017/075243

§ 371 Date: Nov. 3, 2017

Related U.S. Application Data
(60) Provisional application No. 62/404,081, filed on Oct. 4, 2016.

(51) Int. Cl. A23L 19/00; A23L 29/244; A23L 33/105; A61K 31/05; A61K 31/352; A61K 36/28; A23K 10/30

(52) U.S. Cl. CPC A23L 19/01; A23L 29/244; A23L 33/105; A61K 31/05; A61K 31/352; A61K 36/28; A23V 2300/31; A23V 2002/00; A23V 2200/3202; A23V 2250/5062; A23V 2300/10; A23V 2300/24; A23K 10/30

(57) ABSTRACT

This disclosure relates to methods for making a composition of a cannabinoid and chicory flour. According to a method, the composition is manufactured by combining an amount of a cannabinoid and an amount of chicory flour comprising about 10% (w/w) to about 90% (w/w) less 11β,13-dihydrolactucin than a chicory flour made from the taproots of Cichorium intybus.

### BACKGROUND

This disclosure is generally directed towards compositions comprising low bitter chicory flour, as well as syrup and puffs made from low bitter chicory flour, and naturally-occurring cannabinoids, cannabinoid prodrugs and cannabinoid analogs. Cannabinoids are terpenophenolic compounds found in Cannabis sativa, an annual plant belonging to the Cannabaceae family. The plant contains more than 100 cannabinoids which accumulate mainly in the glandular trichomes. The main psychoactive cannabinoid is tetrahydrocannabinol (THC), or more precisely its main isomer (−)-trans-Δ⁹-tetrahydrocannabinol ((6aR,10aR)-Δ⁹-tetrahydrocannabinol), which is used for treating a wide range of medical conditions, including glaucoma, AIDS wasting, neuropathic pain, treatment of spasticity associated with multiple sclerosis, fibromyalgia and chemotherapy-induced nausea. THC is also effective for treating allergies, inflammation, infection, epilepsy, depression, migraine, bipolar disorders, anxiety disorder, drug dependency and drug withdrawal syndromes.

In addition to THC, other biologically active cannabinoids are also present in C. sativa plant. One such cannabinoid is cannabidiol (CBD), which is a potent antioxidant and anti-inflammatory compound known to provide protection against acute and chronic neuro-degeneration. Another biologically active cannabinoid is cannabigerol (CBG). CBG is found in high concentrations in hemp. It is a high affinity α₂-adrenergic receptor agonist, a moderate affinity 5-HT_{1A} receptor antagonist and is a low affinity CB1 receptor antagonist. CBGs are known to possess a mild anti-depressant activity. Cannabichromene (CBC) is another biologically active cannabinoid and is known to possess anti-inflammatory, anti-fungal and anti-viral properties. Tetrahydrocannabivarin (THCV) is known to suppress appetite, improve diabetes and stimulate bone growth. Cannabinol (CBN) is known to induce sleep.

The chicory plant produces large quantities of the prebiotic fiber oligofructose, commonly known as inulin, in the taproot. Chicory (Cichorium intybus) is native to Europe, North Africa, and Western Asia and naturalized in North America. There are many commercial uses of the plant. For instance, chicory roots can be dried and roasted and used as a coffee substitute. Further, the chicons (shoots and leaves) can be used for consumption in salads and vegetable dishes. However, the taproots of chicory are very bitter in taste.

### SUMMARY OF THE INVENTION

The present invention provides food and drink compositions in which the prebiotic fiber oligofructose is combined with one or more naturally-occurring cannabinoids. One or more cannabinoids are combined with an amount of chicory flour comprising about 10% (w/w) to about 90% (w/w) less 11β,13-dihydrolactucin than a chicory flour made from the taproots of Cichorium intybus

The present invention provides food and drink compositions in which the prebiotic fiber oligofructose is combined with one or more naturally-occurring cannabinoids in order to provide one or more biological responses including appetite suppression, induce sleep, reduce inflammation, reduce pain, reduce or eliminate headache, or control diabetes.

The present invention provides a method of making an appetite suppressing composition. The method includes combining an amount of a cannabinoid and an amount of chicory flour comprising about 10% (w/w) to about 90% (w/w) less 11β,13-dihydrolactucin than a chicory flour made from the taproots of Cichorium intybus.

In further embodiments of the present invention an appetite suppressing composition includes an amount of a cannabinoid and an amount of chicory flour. The chicory flour includes about 10% (w/w) to about 90% (w/w) less 11β,13-dihydrolactucin than a chicory flour made from the taproots of Cichorium intybus. In one embodiment, the composition suppresses appetite to a greater extent in a subject receiving the composition than a subject who does not receive the inventive composition, or a subject who receives an equivalent amount of cannabinoid or chicory flour respectively.

In another embodiment of the present invention an appetite suppressing composition includes an amount of a cannabinoid according to Formula IIA or Formula IIB:

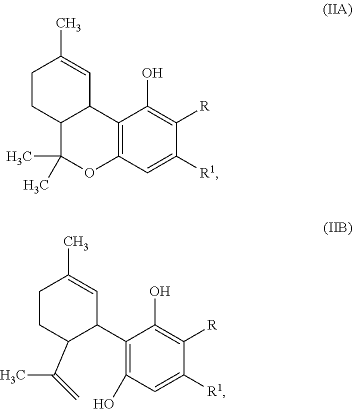

that is produced by a process including contacting a compound according to Formula I:

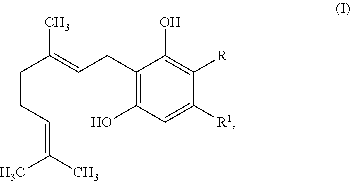

with a cannabinoid synthase in the presence of a solvent to produce the compound according to Formula IIA or Formula IIB. In various embodiments of the present disclosure the variable R is —H, —COOH, or —(CH₂)ₙCOOH. In various embodiments of the present disclosure R¹ is (C₁-C₁₀) alkyl. In various embodiments of the present disclosure the variable n is selected from 1, 2, 3, 4, 5, or 6. The composition further includes an amount of chicory flour comprising about 10% (w/w) to about 90% (w/w) less 11β,13-dihydrolactucin than a chicory flour made from the taproots of Cichorium intybus.

In other embodiments of the invention a composition includes an amount of a cannabinoid and an amount of chicory flour. The chicory flour includes about 10% (w/w) to about 90% (w/w) less 11β,13-dihydrolactucin than a chicory flour made from the taproots of Cichorium intybus.

The invention provides certain advantages. For example, the compositions described herein have a less bitter taste as perceived by a subject compared to a composition made with chicory flour from the taproots of Cichorium intybus. Foods containing chicory flour provide a healthy delivery system for a wide variety of naturally-occurring cannabinoids such as THCA, THC, CBDA, CBD, CBCA, CBC, CBGA, CBG, THCVA, THCV, CBDVA, CBDV, CBCVA, CBCV, CBGVA, CBGV, CBN, CBV, CBNA, CBNVA, CBE, CBL, THV and CBLA.

The naturally-occurring cannabinoids can be incorporated into the food or beverage. Alternatively, one or more cannabinoids can be incorporated into a syrup made from pure inulin, or chicory flour, and the cannabinoid-containing syrup incorporated into the food or beverage, or the syrup is used to coat the food. For example, a health bar, brownie, cookie, candy or the like, can be coated with a cannabinoid-containing syrup, following baking or preparation.

Additionally, administering a composition of the invention suppresses the appetite of a subject to a greater extent compared to the extent of appetite suppression in a subject receiving the same amount of THCVA or THCV or chicory flour. The amount of the cannabinoid in the composition is less than the minimum amount of cannabinoid required to suppress appetite in a subject in the absence of the chicory flour. In various embodiments of the present invention the amount of chicory flour in the composition is less than a minimum amount of chicory flour required to suppress appetite in a subject in the absence of the cannabinoid.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “solvent” as used herein refers to a liquid that can dissolve a solid, liquid, or gas. Non-limiting examples of solvents are silicones, organic compounds, water, alcohols, ionic liquids, and supercritical fluids.

The term “prebiotic” as used herein refers to a non-digestible food ingredient that promotes the growth of beneficial microorganisms in a gastrointestinal system.

The term “tetrahydrocannabinolic acid”, “Δ9-tetrahyrdocannabinoic acid” or “THCA” are used interchangeably. The structure of THCA is represented as:

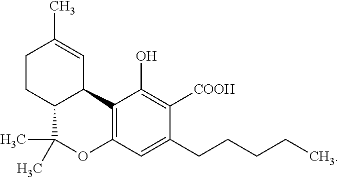 [SMILES: CCCCCc1cc2c(c(O)c1C(=O)O)[C@@H]1C=C(C)CC[C@H]1C(C)(C)O2]

The terms “tetrahycrocannabinol”, “Δ9-tetrahydrocannabinol”, or “THC” are used interchangeably. The structure of THC is represented as:

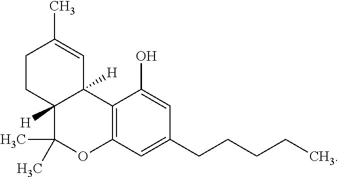 [SMILES: CCCCCc1cc(O)c2c(c1)OC(C)(C)[C@@H]1CCC(C)=C[C@@H]21]

The terms “cannabidiolic acid” or “CBDA” are used interchangeably. The structure of CBDA is represented as:

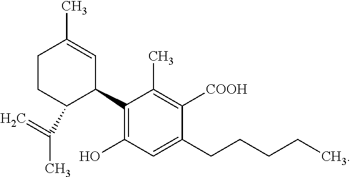 [SMILES: C=C(C)[C@@H]1CCC(C)=C[C@H]1c1c(O)cc(CCCCC)c(C(=O)O)c1C]

The terms “cannabidiolic” or “CBCA” are used interchangeably. The structure of CBC is represented as:

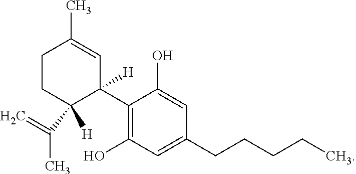 [SMILES: C=C(C)[C@@H]1CCC(C)=C[C@H]1c1c(O)cc(CCCCC)cc1O]

The terms “cannabichromenic acid” or “CBCA” are used interchangeably. The structure of CBCA is represented as:

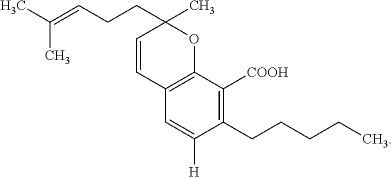 [SMILES: CCCCCc1ccc2c(c1C(=O)O)OC(C)(CCC=C(C)C)C=C2]

The terms “cannabichromene” or “CBC” are used interchangeably. The structure of CBC is represented as:

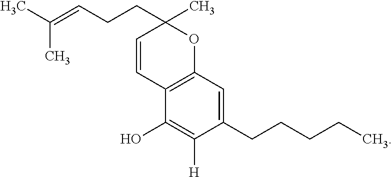 [SMILES: CCCCCc1cc(O)c2c(c1)OC(C)(CCC=C(C)C)C=C2]

The terms “cannabigerolic acid” or “CBGA” are used interchangeably. The structure of CBGA is represented as:

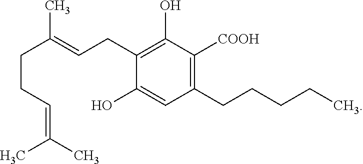 [SMILES: CCCCCc1cc(O)c(C/C=C(\C)CCC=C(C)C)c(O)c1C(=O)O]

The terms “cannabigerol” or “CBG” are used interchangeably. The structure of CBG is represented as:

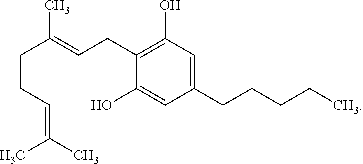 [SMILES: CCCCCc1cc(O)c(C/C=C(\C)CCC=C(C)C)c(O)c1]

The terms “tetrahydrocannabivarinic acid”, “Δ9-tetrahydrocannabivarinic acid”, or “THCVA” are used interchangeably. The structure of THCVA is represented as:

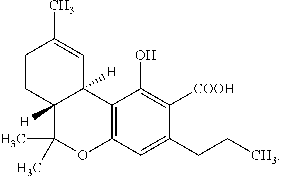 [SMILES: CCCc1cc2c(c(O)c1C(=O)O)[C@@H]1C=C(C)CC[C@H]1C(C)(C)O2]

The terms “tetrahydrocannabivarin”. “tetrahydrocannabivarin, “Δ9-THC-v,” “THC-v”, and “THV” are used interchangeably. The structure of THC-v is represented as:

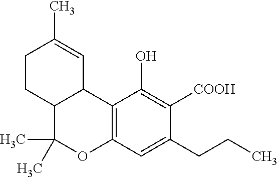 [SMILES: CCCc1cc2c(c(O)c1C(=O)O)C1C=C(C)CCC1C(C)(C)O2]

The terms “cannabivarin”, “cannabivarol”, and “CBV” are used interchangeably and they refer to the oxidized form of THC-v. CBV is a non-psychoactive cannabinoid whose structure is:

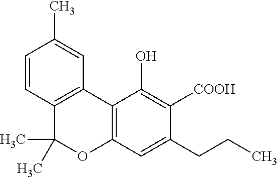 [SMILES: CCCc1cc2c(c(O)c1C(=O)O)-c1cc(C)ccc1C(C)(C)O2]

The terms “cannabidivarinic acid” or “CBDVA” are used interchangeably. The structure of CBDVA is represented as:

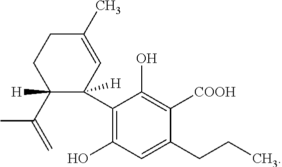 [SMILES: C=C(C)[C@@H]1CCC(C)=C[C@H]1c1c(O)cc(CCC)c(C(=O)O)c1O]

The terms “Cannabidivarin” or “CBDV” are used interchangeably. The structure of CBDV is represented as:

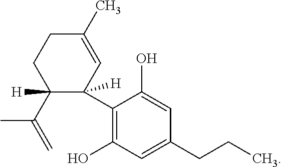 [SMILES: C=C(C)[C@@H]1CCC(C)=C[C@H]1c1c(O)cc(CCC)cc1O]

The terms “cannabichromevarinic acid” or “CBCVA” are used interchangeably. The structure of CBCVA is represented as:

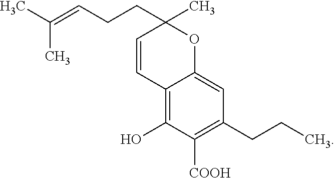 [SMILES: CCCc1cc2c(c(O)c1C(=O)O)C=CC(C)(CCC=C(C)C)O2]

The terms “cannabichromevarin” or “CBCV” are used interchangeably. The structure of CBCV is represented as:

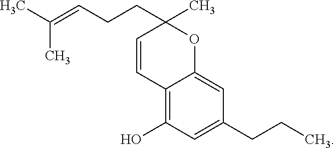 [SMILES: CCCc1cc(O)c2c(c1)OC(C)(CCC=C(C)C)C=C2]

The terms “cannabigerovarinic acid” or “CBGVA” are used interchangeably. The structure of CBGVA is represented as:

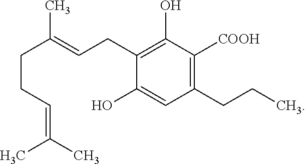 [SMILES: CCCc1cc(O)c(C/C=C(\C)CCC=C(C)C)c(O)c1C(=O)O]

The terms “Cannabigerovarin” or “CBGV” are used interchangeably. The structure of CBGV is represented as:

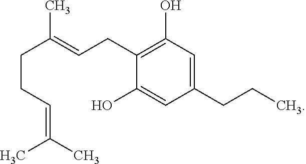 [SMILES: CCCc1cc(O)c(C/C=C(\C)CCC=C(C)C)c(O)c1]

The terms “cannabicyclolic acid” or “CBLA” are used interchangeably. The structure of CBLA is represented as:

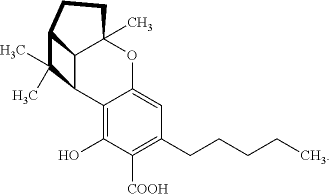

The terms “cannabinol” and “CBN” are used interchangeably and they refer to the oxidized form of THC. Cannabinol is a non-psychoactive cannabinoid whose structure is:

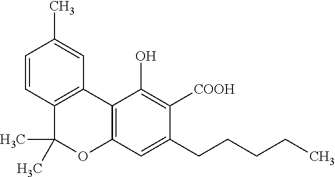 [SMILES: CCCCCc1cc2c(c(O)c1C(=O)O)-c1cc(C)ccc1C(C)(C)O2]

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

### Appetite Suppressing Composition

According to various embodiments of this disclosure, an appetite suppressing composition is a composition of a cannabinoid such as THCVA or THCV and chicory flour. The composition can be a homogenous mixture and can be administered as a stand-alone nutraceutical, incorporated into a food product, or administered as a pharmaceutical composition. The cannabinoid can be one of many different types of cannabinoids that are either naturally occurring, synthetic cannabinoids, or analogs of natural cannabinoids.

The chicory flour includes about 10% (w/w) to about 90% (w/w) less 11β,13-dihydrolactucin than a chicory flour made from the taproots of common chicory (Cichorium intybus). This can result in the chicory flour having a less bitter taste than chicory flour isolated from Cichorium intybus, as perceived by a subject.

The composition has an increased appetite suppressing effect compared to that of the individual components. For example, the amount of composition that suppresses the appetite of a subject to a greater extent can be the same or less than the amount of either cannabinoid or chicory flour individually. Therefore, the amount of the cannabinoid in the disclosed inventive composition can be less than the minimum amount of the cannabinoid required to suppress appetite in a subject in the absence of the chicory flour. Additionally, the amount of chicory flour in the composition can be less than a minimum amount of chicory flour that is required to suppress appetite in a subject in the absence of the cannabinoid. Additionally, the composition can have a greater appetite suppressing effect than a composition that includes only the cannabinoid or a composition that contains only chicory flour in any amount.

In the composition, the amount of the cannabinoid can range from about 0.01% (w/w) to about 20% (w/w), or 1% (w/w) to about 10% (w/w), or less than, or equal to, or greater than, 0.05% (w/w), 0.10, 0.20, 0.30, 0.40, 0.50, 0.60, 0.70, 0.80, 0.90, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.5, 13.0, 13.5, 14.0, 14.5, 15.0, 15.5, 16.0, 16.5, 17.0, 17.5, 18.0, 18.5, 19.0, or 19.5% (w/w) of the composition.

The amount of the chicory flour can range from about 1% (w/w) to about 60% (w/w), or 20% (w/w) to about 40% (w/w), or less than, or equal to, or greater than about 2, 2.5, 3, 3.5, 4, 4.5, 5, 5.5, 6, 6.5, 7, 7.5, 8, 8.5, 9, 9.5, 10, 10.5% (w/w), 11.0, 11.5, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, or 59.5% (w/w). The exact amount of each component can vary depending on the specific cannabinoid or cannabinoids that are included in the composition. The exact amount can also vary depending on the individual human or animal that the composition is administered to. Varying the amount of each component in the composition allows the composition to be customized to control the degree of the appetite suppressing effect of the composition in the individual human or animal.

The appetite suppressing effect of the composition can be determined in many different ways. For example, one method, described briefly herein, can determine the appetite suppressing effect of the composition by administering the composition to a number of human or animal subjects. The amount of food consumed by each test subject subsequent to the administration of the composition can be monitored, recorded, and compared to a control subject that is not given the composition.

Variables that can be changed to further determine the appetite suppressing effect include the amount of the composition that is given to the subject, the time of day the composition is administered, the frequency of administration of the composition, and the time of day and frequency with which the subject is allowed to access food each day. For example, the amount of the composition administered can range from 0.05 mg to about 10 mg. In some experiments, the composition can be administered hourly, twice daily, once daily, or weekly. In other experiments food can be administered hourly or daily. Administration of food can be concurrent with administration of the composition or can be delayed for a period of time such as 1 to 12 hours after the composition is administered. From the gathered data, a comparison between the amount of food consumed by the control subject and those subjects administered the composition can reveal whether the composition has an appetite suppressing effect.

As previously mentioned, the cannabinoid is one of many naturally-occurring cannabinoids, a cannabinoid synthesized chemically, or a cannabinoid that is manufactured by a combination of chemical synthesis and bio-catalysis. For example, some cannabinoids are a compound according to Formula IIA or Formula IIB:

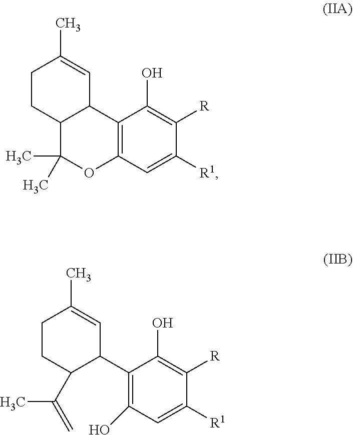

The variable R is chosen from —H, COOH or —(CH₂)ₙCOOH. The variable R¹ is selected from (C₁-C₁₀) alkyl. For example, R¹ is selected from the group consisting of methyl, ethyl, propyl, butyl, pentyl, hexyl, and heptyl. The variable n is selected from 1, 2, 3, 4, 5, or 6. In some embodiments the variable R is —COOH and the variable R¹ is propyl. With R and R¹ defined as such, the compound according to Formula IIA is tetrahydrocannabivarin carboxylic acid (THCVA). In one embodiment, variable R is —COOH, variable R¹ is pentyl and the compound according to Formula IIA is tetrahydrocannabinolic acid (THCA).

Cannabinoids according to Formula IIA can be oxidized to form a compound according to Formula III:

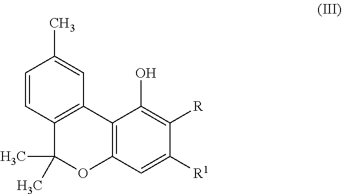

In various embodiments the variable R¹ is pentyl, and the compound according to Formula III is thus Cannabinol (CBN). In various other embodiments the variable R¹ is propyl, and the compound according to Formula III is Cannabivarin (CBV).

As stated above, the cannabinoid can be one of many types of cannabinoids. For example, a composition can include one or more cannabinoids such as Cannabivarin (CBV), Cannabinol (CBN), Cannabigerol (CBG), Cannabigerolic acid (CBGA), Cannabigerovarin (CBGV), Cannabigerovarinic acid (CBGVA), Cannabidiol (CBD), Cannabidiolic acid (CBDA), Cannabidivarin (CBDV), Cannabidivarinic acid (CBDVA), Cannabichromene (CBC), Cannabichromenic acid (CBCA), Cannabichromevarin (CBCV), Cannabichromevarinic acid (CBCVA), Δ9-Tetrahydrocannabinol (THC), Δ9-Tetrahydrocannabinolic acid (THCA), Δ9-Tetrahydrocannabivarin (THCV), Δ9 Tetrahydrocannabivarinic acid (THCVA), Cannabinol (CBN). Cannabinolic acid (CBNA), Cannabivarin (CBNVA), Cannabielsoin (CBE), Cannabicyclol (CBL), Cannabicyclolic Acid (CBLA), and Tetrahydrocannabivarin (THV). Additionally, the composition can include a prodrug of any cannabinoid. The composition can further include any combination of any of the cannabinoids or prodrugs thereof.

The chicory in the composition can be characterized by its components. In addition to the 11β,13-dihydrolactucin content, the chicory can be characterized by its oligofructose content. Oligofructose is a carbohydrate that occurs in many plants and bacteria. Oligofructoses of plant origin typically is a polydisperse composition that includes oligosaccharides and polysaccharides composed of fructose units linked to each other through β(2-1) fructosyl-fructose linkages, and which mostly terminate in one glucose unit. Oligofructose of plant origin usually are composed of linear chains, but may contain some branched chains. Main plant sources for oligofructose are roots of Cichorium intybus and tubers from Dahlia and Jerusalem artichoke, in which oligofructose can be present, respectively, in concentrations of about 15-18%, 12% and 14 to 18% on a fresh weight basis. Oligofructose can be extracted from these plant parts, purified and optionally fractionated to remove impurities, mono- and disaccharides and undesired oligosaccharides, in order to provide various grades of oligofructose. Oligofructose can be generally represented, depending from the terminal carbohydrate unit, by the general formulae GFₙ and Fₘ, wherein G represents a glucose unit, F represents a fructose unit, n is an integer representing the number of fructose units linked to the terminal glucose unit, and m is an integer representing the number of fructose units linked to each other in the carbohydrate chain.

In some embodiments the percentage (w/w) of oligofructose in the chicory flour is equal to or greater than a percentage (w/w) of oligofructose found in taproots of Cichorium intybus. For example, the chicory flour includes about 50% (w/w) to about 90% (w/w) oligofructose, or about 75% (w/w) to about 90% (w/w) oligofructose, or less than, equal to, or greater than about 55% (w/w), 60, 65, 70, 75, 80, and 85% (w/w) oligofructose. Without being bound to any theory, the inventors believe that greater oligofructose content in the chicory flour correlates with greater appetite suppressing characteristics. Therefore, the oligofructose content of the chicory flour can be varied in order to adjust the appetite suppressing characteristics of the composition. For example, chicory flour having a higher oligofructose content can be used regardless of the amount or type of cannabinoid that is included in the composition. Alternatively, chicory flour having higher content of oligofructose can be used if the cannabinoid has relatively weak appetite suppressing characteristics or if a small amount of a cannabinoid having relatively strong appetite suppressing characteristics is used. Further, a chicory flour having a relatively lower content of oligofructose can be used if the cannabinoid in the composition has strong appetite suppressing characteristics.

The chicory flour can include additional components that make up the balance of the mass. For example, the chicory flour can include less than 1% (w/w) of a sugar, less than 5% (w/w) of a fat. The chicory flour can also include about 10% (w/w) to about 20% (w/w) of an insoluble fiber and about 50% (w/w) to about 75% (w/w) of a soluble fiber. Additionally, the chicory flour can include about 2.5% (w/w) to about 10% (w/w) of a protein. The chicory flour optionally includes substantially equivalent amounts (w/w) of 8-deoxylactucin and lactucopicrin as compared to a chicory flour made from the taproots of Cichorium intybus.

A syrup-containing product is one or more of dairy products, nutrition bars, energy bars, breakfast bars, confectionery, bakery products, cookies, biscuits, cereal chips, snack products, breakfast cereals and candies. Syrups can generally be characterized as relatively thick fluids that have a viscosity greater than water. In one aspect of the invention, a food product such as a crisp product comprises at least 5% (w/w) of the low bitter chicory flour. In another aspect of the invention, the crisp product comprises at least 10% (w/w) of total ingredients of the low bitter chicory flour. In yet another aspect of the invention, the crisp product comprises at least 20% (w/w) of total ingredients of the low bitter chicory flour. In a different aspect of the invention, the crisp product comprises at least 30% (w/w) of total ingredients of the low bitter chicory flour. In another aspect of the invention, the crisp product comprises at least 40% (w/w) of total ingredients of the low bitter chicory flour. In a different aspect of the invention, the crisp product comprises between 5% (w/w) and 40% (w/w) of total ingredients of the low bitter chicory flour.

In another embodiment, the invention provides a syrup comprising at least 1.5% (w/w) of total ingredients of the low bitter chicory flour, wherein said flour is less bitter than a flour made from the taproots of the chicory variety ‘Orchies’ as determined in taste test. In one aspect of the invention, the chicory flour comprises from 10% (w/w) to 90% (w/w) less dihydrolactucin compared to a bitter chicory flour made from the taproots of Chicorium intybus. In another aspect of the invention, the chicory flour comprises from 10% (w/w) to 30% (w/w) less dihydro-8-deoxylactucin compared to a bitter chicory flour made from the taproots of Chicorium intybus, and comprises no reduced levels of lactucin, 8-deoxylactucin and lactucopicrin compared to a bitter chicory flour made from the taproots of Chicorium intybus.

In yet another aspect of the invention, the chicory flour contains 50% to 70% oligofrutose with a chain length in the range of about 10 to about 40, and with an average chain length in the range of about 12 to about 20.

In one embodiment, the syrup comprises from 1% (w/w) to 20% (w/w) of total ingredients of the low bitter chicory flour. In a different embodiment, the syrup comprises from 20% (w/w) to 40% (w/w) of total ingredients of the low bitter chicory flour. In a different aspect of the invention, the syrup comprises between 5% (w/w) and 40% (w/w) of total ingredients of the low bitter chicory flour.

In yet another embodiment, the invention provides a method of obtaining a syrup with reduced bitterness, comprising cooking chicory taproots at a temperature between 43° C. and 100° C. drying the chicory taproots after cooking to produce chicory chips, milling the chicory chips to produce chicory flour, sieving the resulting flour and producing a syrup from the flour. In one aspect of the invention, sieving comprises the use of a 30 mesh (0.500 mm), a 40 mesh (0.420 mm), a 45 mesh (0.354 mm), a 50 mesh (0.297 mm), a 60 mesh (0.250 mm), a 70 mesh (0.210 mm), an 80 mesh (0.177 mm), a 100 mesh (0.149 mm) or higher sieve. In another aspect of the invention, cooking comprises one or more of boiling, microwaving, steaming or blanching unroasted chicory taproots. To make the syrup, chicory flour is suspended in water and heated to between 4° C. and 100° C. with stirring. Alternatively, pure oligofrutose syrup is utilized.

One or more cannabinoids are incorporated into chicory fluid, or oligofructuse fluid, while it is hot. One or more of the cannabinoids are formulated as liposomes or micelles and the then liposome or micelle-containing cannabinoids are incorporated into the chicory syrup.

The chicory syrup containing one or more cannabinoids can incorporated into beverages, foods or used to coats pre-made foods such as health bars, cookies, brownies, and the like, to deliver the cannabinoids in a form that is not degraded during the cooking or baking process.

The chicory flour and cannabinoid composition can be administered in many different forms. In some embodiments the composition is incorporated into a food product that is consumable by eating or drinking by a human or animal. A variety of food and beverage products are suitable for this purpose. Non-limiting examples include one or more of dairy products, yogurts, ice creams, milk-based drinks, milk-based garnishes, puddings, milkshakes, egg custard, cheeses, nutrition bars, energy bars, breakfast bars, confectionery, bakery products, crackers, cookies, biscuits, cereal chips, snack products, ice tea, fruit juice, diet drinks, sodas, sports drinks, powdered drink mixtures for dietary supplementation, infant and baby food, calcium-supplemented orange juice, bread, croissants, breakfast cereals, pasta, noodles, spreads, sugar-free biscuits and chocolates, calcium chews, meat products, mayonnaise, salad dressings, nut butter, sauces, syrups and soups. The cannabinoid and chicory flour component of the food can be added as individual components or as a mixture. Additionally the cannabinoid component can be first incorporated into a liposome or micelle, which is then added to the food product. Examples of cannabinoids disposed within liposomes or micelles can be found U.S. Pat. No. 8,808,734, filed on Jul. 7, 2011 and U.S. Pat. No. 9,095,555, filed on Jun. 23, 2014, the contents of both are hereby incorporated by reference.

The chicory flour and cannabinoid food or beverage can be designed to induce sleep by incorporating a proper dose of THC and/or CBN. The chicory flour and cannabinoid food or beverage can be designed with a proper dose of THC to reduce pain. The chicory flour and cannabinoid food or beverage can be designed to reduce inflammation by incorporating a proper does of CBGA and/or CBG and/or CBDA and/or CBD and/or CBCA and/or CBC. The proper dose can be 10 to 999 micrograms, 1-10 milligrams or, 11-100 milligrams, 101-500 milligrams or more than 500 milligrams per serving of the food or beverage, depending on the cannabinoid and its use.

Additionally, the composition can be included in an animal feed product. The food, beverage, or animal feed product can include about 1% (w/w) to about 60% (w/w) of the composition, or about 5% (w/w) to about 55% (w/w), or about 10% (w/w) to about 50% (w/w), or about 15% (w/w) to about 45% (w/w), or about 20% (w/w) to about 40% (w/w), or about 25% (w/w) to about 35% (w/w), or less than, equal to, or greater than about 5% (w/w), 10, 15, 20, 25, 30, 35, 40, 45, 50, or 55% (w/w).

The chicory flour and cannabinoid composition can be included in a pharmaceutical application. That is the composition of a cannabinoid and the disclosed chicory flour can be administered to a patient or subject in need of treatment either alone or in combination with other compounds having similar or different biological activities. For example, the compounds and compositions of the invention are administered in a combination therapy, i.e., either simultaneously in single or separate dosage forms or in separate dosage forms within hours or days of each other.

Thus, the disclosure provides a pharmaceutical composition comprising a cannabinoid and chicory flour or a pharmaceutically acceptable solvate, or stereoisomer of a cannabinoid in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, and flavor imparting agents.

The disclosed composition can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions in accordance with the invention include without limitation tablets, troches, lozenges, aqueous or oily suspensions, dispersible powders or granules, emulsion, hard or soft capsules, syrups or elixirs.

Encompassed within the scope of the disclosure are pharmaceutical compositions suitable for single unit dosages that comprise chicory flour, at least one of tetrahydrocannabivarin (THC-v), cannabinol (CBN), or cannabivarin (CBV), or a pharmaceutically acceptable solvate, or stereoisomer and a pharmaceutically acceptable carrier.

Compositions suitable for oral use may be prepared according to any method known to the art for the manufacture of pharmaceutical compositions. For instance, liquid formulations of a cannabinoid and chicory flour can contain one or more agents selected from the group consisting of sweetening agents, flavoring agents, coloring agents and preserving agents in order to provide pharmaceutically elegant and palatable preparations of the inventive composition.

For tablet compositions, the cannabinoid and chicory flour in admixture with non-toxic pharmaceutically acceptable excipients is used for the manufacture of tablets. Exemplary excipients include without limitation inert diluents, such as calcium carbonate, sodium carbonate, lactose, calcium phosphate or sodium phosphate; granulating and disintegrating agents, such as corn starch, or alginic acid; binding agents, for example starch, gelatin or acacia, and lubricating agents, such as magnesium stearate, stearic acid or talc. The tablets can be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the cannabinoid and chicory flour are mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions the cannabinoid and chicory flour are admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, polyoxyethylene stearate, heptadecaethyleneoxycetanol, polyoxyethylene sorbitol monooleate, polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the cannabinoid and chicory flour in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Syrups and elixirs can be formulated with sweetening agents, for example glycerol, propylene glycol, sorbitol or sucrose. Such formulations may also contain a demulcent, a preservative, and flavoring and coloring agents.

### Methods of Making a Cannabinoid

As stated herein the cannabinoid of the appetite suppressing composition can be extracted from a plant, synthesized chemically, or synthesized using a combination of biological and chemical methods. Illustrative biological methods include without limitation, the use of chemical biology to produce cannabinoids as well as analogs of phytocannabinoids. In one example, chemical biology permits the synthesis of cannabinoids by cloning an enzyme, such as a cannabinoid synthase into a yeast or a bacterial cell. The cannabinoid synthase that is produced can be used to manufacture a phytocannabinoid or an analog of a phytocannabinoid by contacting the enzyme with a suitable substrate. The resulting cannabinoid can be further modified chemically or can be modified biologically. Any one of these methods permits the production of a specific cannabinoid with desired appetite suppressing qualities. A benefit of using chemical biology to synthesize cannabinoids is that this method permits the production of ultra-pure cannabinoids. Additionally, the use of chemical biology, in some circumstances, permits the production of cannabinoids in large quantities, for instance in quantities that that are not economical or practical when using standard extraction methods. Additionally, whether the cannabinoids are synthetically derived or isolated from plants, the opportunity exists to chemically modify the cannabinoid in order to alter or enhance the biological properties of the cannabinoid in the composition. In addition to the methods described herein, the cannabinoids can by synthesized using many other methods. Examples of such methods are described in U.S. Provisional Patent Applications No. 62/303,811, filed on Mar. 3, 2016; No. 62/317,979, filed on Apr. 4, 2016; No. 62/321,471, filed on Apr. 12, 2016; and No. 62/357,766, filed on Jul. 1, 2016, the contents of which are all incorporated herein by reference. Further examples of biological synthesis of cannabinoids can be found in U.S. Pat. No. 9,394,510.

Some cannabinoids for use in the present composition are obtained by contacting a compound according to Formula I:

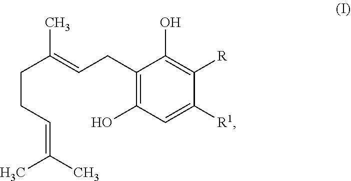

with a cannabinoid synthase in the presence of a solvent. In some embodiments of Formula I the variable R is selected from —H, —COOH, or —(CH₂)ₙCOOH. In some embodiments of Formula I the variable R¹ is selected from the group of methyl, ethyl, propyl, butvl, pentyl, hexyl, and heptyl. In some embodiments of Formula I the variable n is selected from 1, 2, 3, 4, 5, or 6. In some embodiments of the present invention the cannabinoid synthase is tetrahydrocannabinolic acid (THCA) synthase, or a tetrahydrocannabivarin acid (THCVA) synthase, which form a Formula IIA compound. Other cannabinoid synthases include cannabidolic acid (CBDA) synthase, which forms a Formula IIB compound. Compounds according to Formula IIA or Formula IIB are shown below:

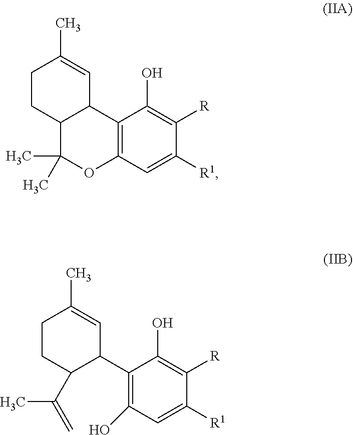

In some embodiments the cannabinoid synthase used is tetrahydrocannabivarin acid (THCVA) synthase, the variable R is —COOH, and the variable R¹ is propyl, thus defining a cannabinoid according to Formula IIA that is tetrahydrocannabivarin acid (THCVA), which can be further isolated and purified prior to being incorporated into the composition. According to another aspect of the disclosure the cannabinoid synthase is tetrahydrocannabinolic acid (THCA) synthase, the variable R is —COOH, and the variable R¹ is pentyl. Accordingly, the cannabinoid according to Formula IIA is Δ9-tetrahydrocannabinolic acid (THCA), which can be further isolated and purified prior to being incorporated into the composition.

According to another aspect of the disclosure, THCVA is decarboxylated to form THC-v, after it is isolated from the reaction mixture. Alternatively, decarboxylation of THCVA to THC-v is carried out prior to isolating and purifying the crude THC-v. Decarboxylating the THCVA can be accomplished by contacting the THCVA with a weak base such as sodium bicarbonate or by exposing the compound to UV-light. In further embodiments the THCA can be decarboxylated to form THC. Decarboxylation can be accomplished by heating a solution of the Formula IIA compound.

The cannabinoid according to Formula IIA can be oxidized prior to or after decarboxylation to produce a cannabinoid according to Formula III:

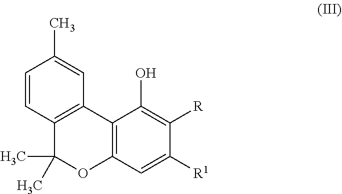

In various embodiments the THC-v is oxidized to produce cannabivarin (CBV). In further embodiments THCA is oxidized by exposure to air or UV-light to provide cannabinolic acid (CBNA). Cannabinol (CBN) can be produced by decarboxylating CBNA. Scheme 1 illustrates the synthesis of THCVA, THC-v and CBV. Scheme 2 illustrates the synthesis of THCA, THC, CBNA, and CBN.

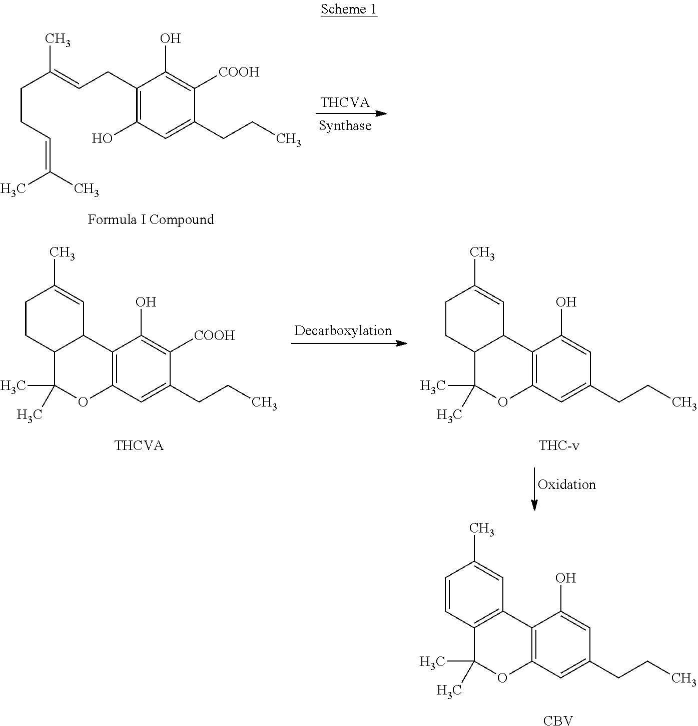

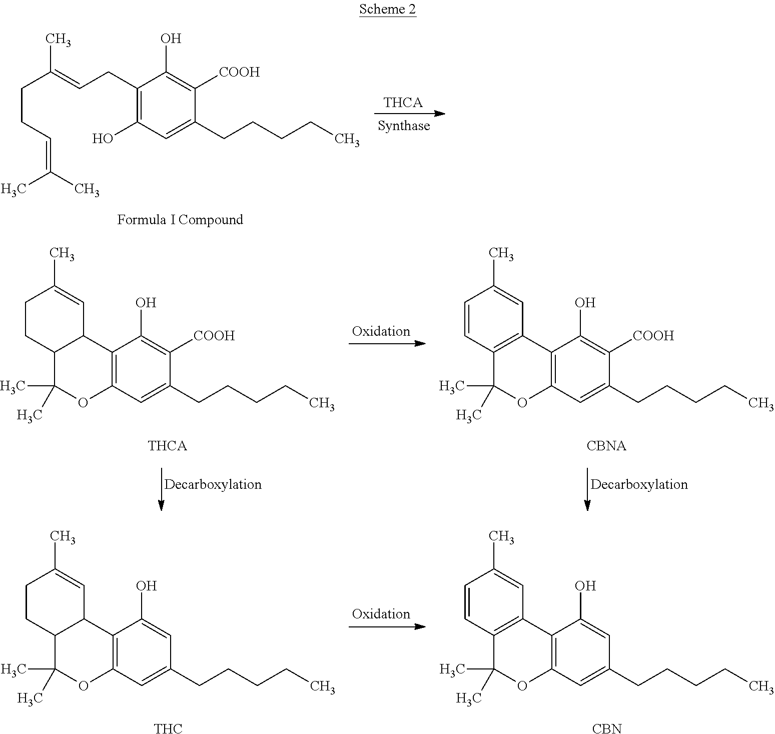

The concentration of non-aqueous solvent in the reaction mixture can have an effect on the rate of the enzyme-catalyzed reaction as well as the yields of CBD, CBV, CBN and THC-v. Exemplary non-aqueous solvents include without limitation, (C₁-C₇) alcohols, (C₁-C₇) alkanes, un-substituted, mono- or di-substituted toluene, and oils such as olive oil, hemp oil. Further, the presence of cyclodextrins and cyclic oligosaccharides that are amphiphilic in nature can accelerate the rate of the enzyme (cannabinoid synthase) catalyzed cyclization of a Formula I compound.

Additionally, the pH of the reaction mixture can influence the ratio of the cannabinoid products produced using the described methods. In one embodiment, when a Formula I compound in which R is —COOH and R¹ is pentyl is contacted with the enzyme THCA synthase the products (Formula IIA compounds) will be a mixture tetrahydrocannabinolic acid (THCA) or cannabichromene acid (CBCA). The ratio of THCA to CBCA formed depends on the pH of the reaction mixture.

In some embodiments, the synthesis of CBV, CBN and THC-v according to the disclosed method is performed at a pH in a range between 3.0 and 8.0, such as at a pH in a range between 3.0 and 7.0, between 3.0 and 6.0, between 3.0 and 5.0, or between 3.0 and 4.0. In one embodiment, the reaction is performed at a pH in a range between 3.8 and 7.2. According to another embodiment, the pH is in a range between 3.5 and 8.0, between 3.5 and 7.5, between 3.5 and 7.0, between 3.5 and 6.5, between 3.5 and 6.0, between 3.5 and 5.5, between 3.5 and 5.0, or between 3.5 and 4.5.

The cannabinoid according to Formula IIA, Formula IIB or Formula III can be purified prior to incorporation in to the composition. Purification is effected by procedures routinely used in the chemical and biochemical arts, including solvent extraction or chromatographic purification methods. The purity of the cannabinoid product is determined by thin layer chromatography, High Performance Liquid Chromatography coupled to a mass spectrometer (HPLC-MS), or by any suitable analytical technique. Nuclear magnetic resonance spectroscopy, mass spectral analysis, or UV, visible spectroscopy, can be used to confirm the identity of the inventive compounds.

Typically, the enantiomeric purity of the compounds described herein is from about 90% ee to about 100% ee. For instance, a cannabinoid according to the present invention can have an enantiomeric purity of about 91% ee, about 92% ee, about 93% ee, about 94% ee, about 95% ee, about 96% ee, about 97% ee, about 98% ee and about 99% ee.

The synthesis of the cannabinoids can be accomplished in a bioreactor that includes the compound according to Formula I, the solvent, and the cannabinoid synthase. The bioreactor can include a control mechanism configured to control at least one condition of the bioreactor. The condition of the bioreactor can be the temperature, solvent, pressure and, pH, and combinations thereof.

### Method of Making Chicory Flour

The chicory flour of the composition includes about 10% (w/w) to about 90% (w/w) less 11β,13-dihydrolactucin than a chicory flour made from the taproots of common chicory (Cichorium intybus). The decrease in the amount of 11β,13-dihydrolactucin causes the chicory flour of the composition to a produce a relatively less bitter taste than that of Cichorium intybus, as perceived by a subject. Therefore, the chicory flour of the present disclosure can be more palatable to a subject than a composition of a cannabinoid and a chicory flour made from the taproots of Cichorium intybus. The chicory flour in the disclosed composition, however, is not naturally occurring. A method will now be described for producing the instant chicory powder from the taproots of Cichorium intybus or any other variety of chicory.

As a non-limiting example the method can include cooking chicory taproots at a temperature ranging from about 40° C. to about 100° C., or from about 60° C. to about 80° C., or less than, equal to, or greater than about 45° C., 50, 55, 60, 65, 70, 75, 80, 85, 90, or 95° C. Cooking the chicory taproots can include boiling, microwaving, steaming, blanching unroasted chicory taproots, or any combination thereof. The chicory roots can be blanched at a temperature ranging from about 60° C. to about 70° C. or less than, equal to, or greater than about 61.0° C. 61.5, 62.0, 62.5, 63.0, 63.5, 64.0, 64.5, 65.0, 65.5, 66.0, 66.5, 67.0, 67.5, 68.0, 68.5, 69.0, or 69.5° C. Cooking is performed to remove the bitter taste by extracting 11β,13-dihydrolactucin from the taproot.

The method can further include drying the cooked chicory taproots, to produce chicory chips. Drying the chicory roots can be accomplished in many ways. For example, the chicory roots can be dried in a furnace at a temperature ranging from about 145° C. to about 180° C., or about 150° C. to about 165° C., or less than, equal to, or greater than about 150° C., 155, 160, 165, 170, and 175° C. The chicory roots can additionally be dried in a fluidized bed dryer.

After the chicory roots have been dried, the chicory roots can be milled to produce the chicory flour. Milling includes the use of hammer mills, universal mills, pin mills, cutting mills, crushers, mechanical mills with internal classifier, high-compression and table roller mills, jet mills, dry media mills and wet media mills, cryogenic mills, colloid mill, ball mills, impact mills, stirred mills, screen mills, drum mills, high-compression roller and table roller mills, jet, dry-media, wet-media mills, and any combination thereof.

### EXAMPLES

Various embodiments of the present invention can be further understood by reference to the following Examples that are offered to illustrate additional embodiments of the invention. The invention is not limited to the Examples given herein.

### Example 1: Determination of Appetite Suppressing Effect by Changing Composition Components

An example of the disclosed composition is one including THC-v and a chicory flour having about 10% (w/w) to about 90% (w/w) less 11β,13-dihydrolactucin than a chicory flour made from the taproots of Cichorium intybus. The chicory flour includes less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 10% (w/w) of a protein. The chicory flour further includes a percentage (w/w) of oligofructose that is equal to a percentage (w/w) of oligofructose in the taproots of Cichorium intybus. The composition comprises about 20 mg THC-v and about 60 mg chicory flour.

THC-v and chicory individually have appetite suppressing properties. However, a composition having both can show an increased appetite suppressing effect. Additionally, because the flour includes about 10% (w/w) to about 90% (w/w) less 11β,13-dihydrolactucin than a chicory flour made from the taproots of Cichorium intybus, the characteristic bitter taste of the chicory, as perceived by a subject, is reduced thus making the composition or a product into which the composition is incorporated more palatable to the subject.

The degree to which the composition suppresses the appetite of the subject is determined by quantifying the weight of food consumed by each subject subsequent to the administration of the composition. The subject is an ob/ob mouse, which is available for example from the Jackson Laboratory. The subjects are distributed into four groups. The first group of subjects are given the composition of 20 mg of THC-v and 60 mg of chicory flour. The second group of subjects are given a composition 20 mg of the THC-v and 60 mg of mouse chow filler. The third group of subjects are given a composition of 60 mg of chicory flour and 20 mg of mouse chow filler. The fourth group of subjects is a control group and is given 80 mg of a placebo, which is mouse chow.

The subjects are administered their respective composition once every 24 hours by oral gavage with the first administration occurring at time zero. An equal weight of food is placed in the subject's surroundings in a location that is accessible to the subject three times at 8 hour intervals beginning at the time the composition is administered to the subject. Eight hours after the last amount of food is placed in the subject's surroundings, the subjects are given another administration of the composition and any remaining food in their surroundings is removed. Water is supplied ad libitum. The weight of food remaining from each subject's surroundings is compared to the total weight of food provided to the subject to determine how much food was consumed. At the end of the experiment, a ratio of the total weight of the remaining food to the total weight of food given for each experimental group of mice is calculated. The ratio is highest for the group receiving the composition of THC-v and chicory flour, thereby demonstrating the appetite suppressing effect of the composition.

### Example 2: Determination of Appetite Suppressing Effect by Changing Administration Time of Compositions of Example 1

An example of the disclosed composition is one including THC-v and a chicory flour having about 10% (w/w) to about 90% (w/w) less 11β,13-dihydrolactucin than a chicory flour made from the taproots of Cichorium intybus. The chicory flour includes less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 100% (w/w) of a protein. The chicory flour further includes a percentage (w/w) of oligofructose that is equal to a percentage (w/w) of oligofructose in the taproots of Cichorium intybus. The composition comprises about 20 mg THC-v and about 60 mg chicory flour.

The degree to which the composition suppresses the appetite of the subject is determined by quantifying the weight amount of food consumed by each subject subsequent to the administration of the composition. The subject is an ob/ob mouse. The subjects are distributed into four groups. The first group of subjects is given a 50 mg of a composition of THC-v and chicory flour once every 48 hours. The second group of subjects are given 50 mg of a composition of THC-v and chicory flour once every 24 hours. The third group of subjects are given 50 mg of a composition of the THC-v and chicory flour once every 12 hours. The fourth group of subjects is a control group and is given 50 mg of a placebo mixture of components that do not include THC-v or chicory flour. This study was performed for seven days.

An equal weight amount of food is placed in the subject's surroundings in a location that is accessible to the subject immediately after the composition, or placebo is first administered to the subject. Twenty four hours after the last weight amount of food is placed in the subject's surroundings any remaining food in the surroundings is removed and weighed. Water is supplied ad libitum. At the end of the experiment, the total weight of food remaining from each subject's surroundings is compared to the total weight of food provided to the subject to determine how much food was consumed. The weight of food remaining from each subject's surroundings is compared to the total weight of food provided to the subject to determine how much food was consumed. At the end of the experiment, a ratio of the total weight of the remaining food to the total weight of food given for each experimental group of mice is calculated. The ratio is highest for the third group, i.e., the test group receiving the composition of THC-v and chicory flour most frequently, and ratios for the second and first groups are second and third lowest, respectively, thereby demonstrating the effect of the frequency that the composition is administered to the subjects.

### Example 3: Determination of Appetite Suppressing Effect by Changing Dosage of the THC-v and Chicory Flour in a Composition

An example of the disclosed composition is one including THC-v and a chicory flour having about 10% (w/w) to about 90% (w/w) less 11β,13-dihydrolactucin than a chicory flour made from the taproots of Cichorium intybus. The chicory flour includes less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 10% (w/w) of a protein. The chicory flour further includes a percentage (w/w) of oligofructose that is equal to a percentage (w/w) of oligofructose in the taproots of Cichorium intybus. The composition comprises about 20% (w/w) THC-v and about 60% (w/w) chicory flour.

The degree to which the composition suppresses the appetite of the subject is determined by quantifying the weight amount of food consumed by each subject subsequent to the administration of the composition. The subject is an ob/ob mouse. The subjects are distributed into four groups. Each of the four groups receives a predetermined weight of the THC-v and chicory flour composition. The first group of subjects are given 0.5 g of the composition. The second group of subjects are given 1 g of the composition. The third group of subjects are given 1.5 g of the composition. The fourth group of subjects is a control group and is given a placebo of mouse chow.

Each group of subjects is administered their respective composition once every 24 hours by oral gavage beginning at time zero. An equal weight amount of food is placed in the subjects' surroundings in a location that is accessible to the subject three times at 8 hour intervals beginning 8 hours after the composition is administered to the subject. Eight hours after the last weight of food has been placed in the subject's surroundings, the remaining food is removed and weighed and another weight of food is supplied. Water is supplied to the subject's ad libitum. The weight of food remaining from each subject's surroundings is compared to the total weight of food provided to the subject to determine how much food was consumed. At the end of the experiment, a ratio of the total weight of the remaining food to the total weight of food given for each experimental group of mice is calculated. The ratio is highest for the group receiving the highest dose of the composition of THC-v and chicory flour, i.e., the third test group, and ratios are second and third lowest for the second and first test groups, respectively, thereby demonstrating composition dose-dependence of appetite suppression.

### Example 4: Chocolate Chip Cookie Recipe Made with Composition of THC-v and Chicory Flour

Ingredients: 1 cup butter, softened; 1 cup white sugar. 1 cup packed brown sugar; 2 eggs; 2 teaspoons vanilla extract; 1½ cup of a composition comprising about 20 mg THC-v and about 60 mg chicory flour. 1½ cups all-purpose flour; 1 teaspoon baking soda; 2 teaspoons warm water; ½ teaspoon salt; 2 cups semisweet chocolate chips. Pan size: 18×26-inch sheet pan. Each portion: 1 20 g. cookies.

Preheat oven to 350 degrees F. (175 degrees C.). Sift all-purpose flour, baking soda, and salt. Set aside. Cream the butter in mixer bowl, gradually add the white sugar and brown sugar until smooth. Mix at medium speed for about 3 minutes or until light and fluffy. Combine the eggs with the warm water and gradually add to the creamed mixture. Blend thoroughly about 1 minute, then stir in the vanilla. Mix thoroughly. Gradually add the THC-v and chicory flour. Mix only until the ingredients are combined (about 1 minute), then add the flour, salt and baking soda mix, and mix to combine. Stir in the chocolate chips. Mix on low speed about 1 minute or until evenly distributed. Use #50 scoop to place the cookie dough about 3 inches apart-on a parchment paper that covers an ungreased pan. Bake for about 9-11 minutes in the preheated oven at 375° F., or until edges are nicely browned. Allow to cool, then pack for freezing either in bulk or in individual bags. Instead of being baked, all or portion of the dough can be set aside and consumed directly or frozen for later use.

### Example 5: Brownie Mix Recipe Made with Composition of THC-v and Chicory Flour

Ingredients: 1 cup white sugar; ⅓ cup unsweetened cocoa powder; ¼ cup all-purpose flour; ¼ cup of a composition comprising about 20 mg THC-v and about 60 mg chicory flour; ¼ teaspoon salt; ¼ teaspoon baking powder; ⅓ cup oil; ¼ cup water; 2 eggs; 1 teaspoon vanilla.

Sift together all dry ingredients. In a medium bowl, beat the eggs with the vanilla. Add in and gently stir dry ingredients. Pour the oil and the water into brownie mixture and mix until just blended. Package in 350 gram packs for individual mixes for 8×8 pan of brownies and store in freezer. When ready to bake, pour batter into a greased 8×8 inch pan sprayed with cooking spray and bake in preheated oven at 350° F. for 25 minutes or until done in center.

Alternatively, THC-v is dissolved in oligofructose syrup and the syrup applied to the surface of the baked brownie. The THC-v may in incorporated into the oligofructose in the form of a liposome or micelle.

### Example 6: Pasta Recipe Made with Composition of THC-v and Chicory Flour

Ingredients: 1½ cup all-purpose flour; 1½ cup of a composition comprising about 20 mg THC-v and about 60 mg chicory flour; and 1 cup water.

Mix all ingredients in pasta extruder; extrude into desired shape; dry in air for 30-60 minutes; cook in salted boiling water; drain and mix with desired sauce.

### Example 7: Tortilla Recipe Made with Composition of THC-v and Chicory

Flour

Ingredients: 2 cups all-purpose flour; 2 cups of a composition comprising about 20 mg THC-v and about 60 mg chicory flour; 1 teaspoon salt; 2 teaspoons baking powder; 2 tablespoons oil; and 1½ cups lukewarm water.

Whisk the flour, salt, and baking powder together in a mixing bowl. Mix in the oil and the water until the dough comes together and change to dough hook. Knead/Mix for 5 minutes. Portion into 50 gram dough balls, cover and let sit for 5 to 10 minutes. Set Tortilla Press to 300° F. When pressing, place the dough ball in the center, and press until it whistles (usually 7-9 seconds).

### Example 8: Soft Bread Stick Recipe Made with Composition of THC-v and Chicory Flour

Ingredients: 1½ cups all-purpose flour; 1½ cups of a composition comprising about 20 mg THC-v and about 60 mg chicory flour; 1 teaspoon salt; 2 teaspoons dry yeast powder; 1 teaspoon sugar; 4 tablespoons oil; and 1½ cups lukewarm water.

In large bowl dissolve yeast and sugar in water (110° F.) and let sit 5 minutes. Add oil and gradually add the all-purpose flour first and then a composition comprising about 20 mg THC-v and about 60 mg chicory flour. Knead for 5 minutes. Place dough into lightly oiled bowl with room to rise double and let sit for at least 1 hour at 110° F. After the dough rises, punch down dough, divide in half, and using sheeter, sheet to 3 inches wide and 2.5 thick. Cut into 5 inch rectangles and roll into a stick. Place about ½ inch apart, lay with seam on the bottom, cover lightly with plastic wrap, then allow to raise for 30 minutes at 110° F. Bake in pre-heated oven at 400° F. for 12-15 minutes until just starting to brown. Brush with melted butter on tops and bottoms.

Various foods can be made using the composition of THC-v and chicory flour in which the THC-v component is disposed within a liposome suspension or a micelle suspension. Examples of foods prepared include the following.

### Example 9: General Protocol for the Manufacture of Liposomes

Pure Cannabinoids, such as THC-v, or cannabinoid extracts are dissolved in a water miscible organic solvent. To this solution various amphipathic molecules such as phospholipids, sterols, and/or fatty acids are also dissolved. This solution containing the cannabinoids and amphipathic molecules is rapidly mixed or injected into an appropriate aqueous environment. The rapid dissolution of the organic solvent causes the dissolved cannabinoids and amphipathic molecules to spontaneously form ordered lipid layers which self-assemble into micro or nano liposomal particles. The size, composition and concentration of the liposomes are controlled by the chemical properties and relative concentrations of the cannabinoids and lipids, the organic solvent/aqueous environment and the physical parameters of the solvent addition. After formation, the liposomes can be processed for consumption by removing at least a portion of any remaining organic solvent(s). This is accomplished, for example, by heating the liposomes, optionally under reduced pressure, to a temperature where the solvent(s) volatilize. The processed liposomes can be freeze dried for storage or for direct incorporation into a consumable product.

In an exemplary composition, the hydrophobic/lipophilic membrane comprises about 40% phosphatidylcholine, about 3.5% phosphatidylethanolamine, about 6% phosphonophospholipids, and about 0.5% of other phospholipids. According to another embodiment the hydrophobic/lipophilic membrane of liposomes in the inventive composition comprises about 26° % phosphatidylcholine, about 10% phosphatidylethanolamine, about 13% phosphonophospholipids, and about 1% of other phospholipids.

### Example 10: General Protocol for the Manufacture of Micelles

A cannabinoid, such as THC-v, its analog or cannabinoid extract are dissolved in a water miscible organic solvent. This solvent cannabinoid solution is rapidly mixed or injected into an appropriate aqueous environment. The rapid dissolution of the organic solvent causes the dissolved cannabinoids to self-align into micro or nano micellar particles. Size, composition and concentration of the micelles are controlled by the chemical properties of the cannabinoids, the organic solvent/aqueous environment and the physical parameters of the solvent addition.

After formation, the micelles can be processed for consumption by removing at least a portion of any remaining organic solvent. This can be accomplished, for instance, by heating the micelles, optionally under reduced pressure, to a temperature where the solvents will volatilize. The liposomes can be immediately freeze dried for storage or for direct incorporation into a consumable product.

### Example 11: Protein Shake Recipe Made with Composition of THC-v in a Liposome Suspension and Chicory Flour

Ingredients: 2 teaspoons of protein powder; 0.4 mL of a liposomal composition of THC-v as described above having a concentration of 50 mg/mL, formed according to the protocol of Example 9, and 60 mg of chicory flour; 1 cup of milk; 2 cups of strawberries; and 2 cups of ice cubes. Blend all contents together.

### Example 12: Protein Shake Recipe Made with Composition of CBD in a Micelle Suspension and Chicory Flour

Ingredients: 2 teaspoons of protein powder; 0.4 mL of a micellar composition of CBD as described above having a concentration of 50 mg/mL, formed according to the protocol of Example 10, and 60 mg of chicory flour; 1 cup of milk; 2 cups of strawberries; and 2 cups of ice cubes. Blend all contents together.

### Example 13: Chocolate Chip Cookie Dough Recipe Made Using a Liposomal Composition of CBD and Chicory Flour

Ingredients 0.4 mg of a composition of CBD in a liposomal suspension having a concentration of 50 mg/mL formed according to the protocol of Example 10 and 60 mg of chicory flour. The ingredients further include ¾ cup of granulated sugar. ¾ cup of packed brown sugar, 1 cup of butter, 1 teaspoon of vanilla, 1 egg, 2 cups of flour, 1 teaspoon of salt, and 12 ounces of chocolate chips. Mix all contents together. The dough is incorporated into foodstuffs such as ice cream.

### Additional Embodiments

The following exemplary embodiments are provided, the numbering of which is not to be construed as designating levels of importance:

Embodiment 1 provides a method of making an appetite suppressing composition comprising (i) combining:
(A) an amount of a cannabinoid; and
(B) an amount of chicory flour comprising about 10% (w/w) to about 90% (w/w) less 11β,13-dihydrolactucin than a chicory flour made from the taproots of Cichorium intybus.

Embodiment 2 provides the method of Embodiment 1, wherein the cannabinoid is a formula according to Formula IIA or Formula IIB:

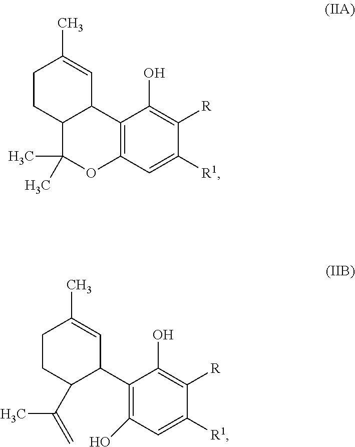

and wherein the method further comprises:
(ii) contacting a compound according to Formula I:

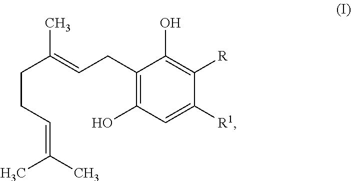

with a cannabinoid synthase in the presence of a solvent to produce the compound according to Formula II
wherein:
R is —H, COOH, or —(CH₂)ₙCOOH;
R¹ (C₁-C₁₀) alkyl; and
n is 1, 2, 3, 4, 5, or 6.

Embodiment 3 provides the method of Embodiment 2, wherein the cannabinoid synthase is tetrahydrocannabinolic acid (THCA) synthase tetrahydrocannabivarin acid (THCVA) synthase, or cannabidolic acid (CBDA) synthase.

Embodiment 4 provides the method of Embodiment 2, wherein R is —COOH and R¹ is propyl.

Embodiment 5 provides the method of Embodiment 3, wherein the compound according to Formula IIA is tetrahydrocannabivarin carboxylic acid (THCVA).

Embodiment 6 provides the method of Embodiment 4, further comprising:
(iii) decarboxylating THCVA to produce THC-v.

Embodiment 7 provides the method of Embodiment 2, further comprising:
(iii′) oxidizing the Formula IIA compound to produce a compound according to Formula III:

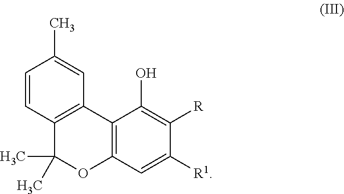

Embodiment 8 provides the method of Embodiment 7, wherein R is —COOH and R¹ is propyl or pentyl.

Embodiment 9 provides the method of Embodiment 8, further comprising the step of decarboxylation by heating a solution of the Formula IIA compound or by exposing a solution of the Formula IIA compound to UV-light.

Embodiment 10 provides the method of Embodiment 8, wherein R¹ is pentyl, and the compound according to Formula III is Cannabinol (CBN).

Embodiment 11 provides the method of Embodiment 7, wherein R¹ is propyl, and the compound according to Formula III is Cannabivarin (CBV).

Embodiment 12 provides the method of Embodiment 1, wherein the cannabinoid is selected from the group consisting of Cannabivarin (CBV). Cannabinol (CBN), Cannabigerol (CBG), Cannabigerolic acid (CBGA), Cannabigerovarin (CBGV), Cannabigerovarinic acid (CBGVA), Cannabidiol (CBD), Cannabidiolic acid (CBDA), Cannabidivarin (CBDV), Cannabidivarinic acid (CBDVA), Cannabichromene (CBC), Cannabichromenic acid (CBCA), Cannabichromevarin (CBCV), Cannabichromevarinic acid (CBCVA), Δ9-Tetrahydrocannabinol (THC), Δ9-Tetrahydrocannabinolic acid (Δ9-THCA), Δ9-Tetrahydrocannabivarin (THCV), Δ9 Tetrahydrocannabivarinic acid (THCVA), Cannabinol (CBN), Cannabinolic acid (CBNA), Cannabivarin (CBNVA), Cannabielsoin (CBE), Cannabicyclol (CBL), Cannabicyclolic Acid (CBLA), Tetrahydrocannabivarin (THV), a prodrug of any cannabinoid thereof, and any combination thereof.

Embodiment 13 provides the method of any one of Embodiments 1-12, further comprising:
(iv) cooking chicory taproots at a temperature ranging from about 40° C. to about 100° C.;
(v) drying the chicory taproots to produce chicory chips; and
(vi) milling the chicory chips to produce the chicory flour.

Embodiment 14 provides the method of Embodiment 13, wherein cooking the chicory taproots comprises one or more of boiling, microwaving, steaming, and blanching unroasted chicory taproots.

Embodiment 15 provides the method of Embodiment 14, wherein the chicory taproots are blanched at a temperature ranging from about 60° C. to about 70° C.

Embodiment 16 provides the method of Embodiment 13, wherein the chicory root chips are dried in a furnace at a temperature ranging from about 145° C. to about 180° C., or in a fluidized bed dryer.

Embodiment 17 provides the method of any one of Embodiments 1-16, wherein the composition comprises about 0.01% (w/w) to about 20% (w/w) cannabinoid.

Embodiment 18 provides the method of any one of Embodiments 1-17, wherein the composition comprises about 1% (w/w) to about 60% (w/w) chicory flour.

Embodiment 19 provides the method of any one of Embodiments 1-18, wherein, the chicory flour comprises less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 10% (w/w) of a protein.

Embodiment 20 provides the method of any one of Embodiments 1-19, wherein the chicory flour includes substantially equivalent amounts (w/w) of 8-deoxylactucin and lactucopicrin compared to a chicory flour made from the taproots of Cichorium intybus.

Embodiment 21 provides the method of any one of Embodiments 2-20, wherein the compound according to Formula 1 is located within a bioreactor containing the solvent and the cannabinoid synthase.

Embodiment 22 provides the method of Embodiment 21, wherein the bioreactor comprises:
a control mechanism configured to control at least one condition of the bioreactor.

Embodiment 23 provides the method of Embodiment 22, wherein the condition of the bioreactor is selected from temperature, solvent, pressure and, pH, and combinations thereof.

Embodiment 24 provides the method of claim 6, wherein decarboxylating the THCVA includes contacting the THCVA with a weak base.

Embodiment 25 provides the method of Embodiment 24, wherein the weak base is sodium bicarbonate.

Embodiment 26 provides the method of Embodiment any one of Embodiments 1-25, wherein the composition is a syrup.

Embodiment 27 provides the method of any one of Embodiments 1-26, wherein the composition is included in a food product.

Embodiment 28 provides the method of Embodiment 27, wherein the food product is chosen from one or more of a dairy product, a yoghurt, an ice cream, a milk-based drink, a milk-based garnish, a pudding, a milkshake, an egg custard, a cheese, a nutrition bar, an energy bar, a breakfast bar, a confectionery, a bakery product, a cracker, a cookie, a biscuit, a cereal chip, a snack product, an ice tea, a fruit juice, a diet drink, a soda, a sports drink, a powdered drink mixture for dietary supplementation, an infant and baby food, a calcium-supplemented orange juice, a bread, a croissants, a breakfast cereal, a pasta, a noodle, a spread, a sugar-free biscuit, a sugar free chocolate, a calcium chew, a meat product, a mayonnaise, a salad dressing, a nut butter, a sauce, a syrup, a crisp, a puff, a candy, and a soup.

Embodiment 29 provides the method of any one of Embodiments 27 or 28, wherein the syrup is a first food product that at least partially coats an external surface of a second food product.

Embodiment 30 provides the method of any one of Embodiments 27-29, wherein the syrup is a first food product and is at least partially dispersed within a second food product.

Embodiment 31 provides the method of Embodiment 1, wherein and the chicory flour is located a food product and the amount of the cannabinoid is dissolved in a syrup comprising an oligofructose.

Embodiment 32 provides the method of Embodiment 31, wherein the syrup comprising the oligofructose is applied to an external surface of the food.

Embodiment 33 provides the method of Embodiment 31, wherein the oligofructose is inulin.

Embodiment 34 provides the method of any one of Embodiments 1-33, wherein the composition is included in an animal feed product.

Embodiment 35 provides the method of any one of Embodiments 1-34, wherein the chicory flour comprises about 50% (w/w) to about 906 (w/w) oligofructose.

Embodiment 36 provides the method of any one of Embodiments 1-35, wherein the chicory flour comprises a percentage (w/w) of oligofructose that is equal to or greater than a percentage (w/w) of oligofructose in the taproots of Cichorium intybus.

Embodiment 37 provides an appetite suppressing composition comprising:
(A) an amount of a cannabinoid; and
(B) an amount of chicory flour comprising about 10% (w/w) to about 90% (w/w) less 11β,13-dihydrolactucin than a chicory flour made from the taproots of Cichorium intybus;
   wherein an amount of composition suppresses the appetite of a subject who is administered the composition to a greater extent than of a subject who is administered the same amount of the cannabinoid or chicory flour.

Embodiment 38 provides the appetite suppressing composition of Embodiment 37, wherein the cannabinoid is a compound according to Formula IIA or Formula IIB:

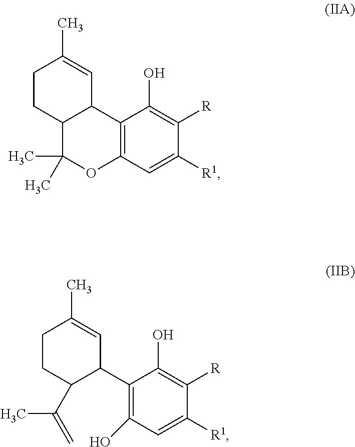

and wherein the compound of Formula IIA is formed from a method comprising:
(i) contacting a compound according to Formula I:

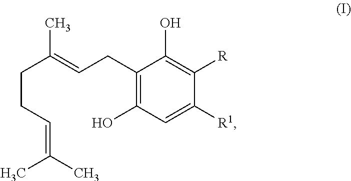

with a cannabinoid synthase in the presence of a solvent to produce the compound according to Formula II
wherein:
R is —H, COOH, or —(CH₂)ₙCOOH;
R¹ (C₁-C₁₀) alkyl; and
n is 1, 2, 3, 4, 5, or 6.

Embodiment 39 provides the appetite suppressing composition of Embodiment 38, wherein the cannabinoid synthase is tetrahydrocannabinolic acid (THCA) synthase tetrahydrocannabivarin acid (THCVA) synthase, or cannabidolic acid (CBDA) synthase.

Embodiment 40 provides the appetite suppressing composition of Embodiment 38, wherein R is —COOH and R¹ is propyl.

Embodiment 41 provides the appetite suppressing composition of Embodiment 40, wherein the compound according to Formula IIA is tetrahydrocannabivarin carboxylic acid (THCVA).

Embodiment 42 provides the appetite suppressing composition of Embodiment 41, further comprising:
(ii) decarboxylating THCVA to produce THC-v.

Embodiment 43 provides the appetite suppressing composition of Embodiment 38, further comprising:
(ii′) oxidizing the Formula IIA compound to produce a compound according to Formula III:

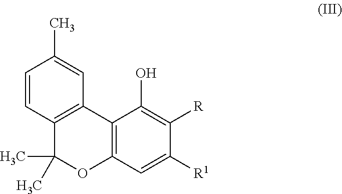

Embodiment 44 provides the appetite suppressing composition of Embodiment 38, wherein R is —COOH and R¹ is propyl or pentyl.

Embodiment 45 provides the appetite suppressing composition of Embodiment 38, further comprising the step of decarboxylation by heating a solution of the Formula IIA compound or by exposing a solution of the Formula IIA compound to UV-light.

Embodiment 46 provides the appetite suppressing composition of Embodiment 43, wherein R¹ is pentyl, and the compound according to Formula III is Cannabinol (CBN).

Embodiment 47 provides the appetite suppressing composition of Embodiment 43, wherein R¹ is propyl, and the compound according to Formula III is Cannabivarin (CBV).

Embodiment 48 provides the appetite suppressing composition of Embodiment 37, wherein the cannabinoid is selected from the group consisting of Cannabivarin (CBV), Cannabinol (CBN), Cannabigerol (CBG), Cannabigerolic acid (CBGA), Cannabigerovarin (CBGV), Cannabigerovarinic acid (CBGVA), Cannabidiol (CBD), Cannabidiolic acid (CBDA), Cannabidivarin (CBDV), Cannabidivarinic acid (CBDVA), Cannabichromene (CBC), Cannabichromenic acid (CBCA), Cannabichromevarin (CBCV), Cannabichromevarinic acid (CBCVA), Δ9-Tetrahydrocannabinol (THC), Δ9-Tetrahydrocannabinolic acid (THCA), Δ9-Tetrahydrocannabivarin (THCV), Δ9 Tetrahydrocannabivarinic acid (THCVA). Cannabinol (CBN), Cannabinolic acid (CBNA), Cannabivarin (CBNVA), Cannabielsoin (CBE), Cannabicyclol (CBL), Cannabicyclolic Acid (CBLA), Tetrahydrocannabivarin (THV), a prodrug of any cannabinoid thereof, and any combination thereof.

Embodiment 49 provides the appetite suppressing composition of any one of Embodiments 37-48, wherein the chicory flour is produced by a process comprising:
(iii) cooking chicory taproots at a temperature ranging from about 40° C. to about 100° C.;
(iv) drying the chicory taproots to produce chicory chips: and
(v) milling the chicory chips to produce the chicory flour.

Embodiment 50 provides the appetite suppressing composition of Embodiment 49, wherein cooking the chicory taproots comprises one or more of boiling, microwaving, steaming, and blanching unroasted chicory taproots.

Embodiment 51 provides the appetite suppressing composition of Embodiment 50, wherein the chicory taproots are blanched at a temperature ranging from about 60° C. to about 70° C.

Embodiment 52 provides the appetite suppressing composition of Embodiment 49, wherein the chicory root chips are dried in a furnace at a temperature ranging from about 145° C. to about 180° C. or in a fluidized bed dryer.

Embodiment 53 provides the appetite suppressing composition of any one of Embodiments 37-52, wherein the composition comprises about 0.01% (w/w) to about 20% (w/w) the cannabinoid.

Embodiment 54 provides the appetite suppressing composition of any one of Embodiments 37-53, wherein the composition comprises about 1% (w/w) to about 60% (w/w) chicory flour.

Embodiment 55 provides the appetite suppressing composition of any one of Embodiments 37-54, wherein, the chicory flour comprises less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 10% (w/w) of a protein.

Embodiment 56 provides the appetite suppressing composition of any one of Embodiments 37-55, wherein the chicory flour includes substantially equivalent amounts (w/w) of 8-deoxylactucin and lactucopicrin compared to a chicory flour made from the taproots of Cichorium intybus.

Embodiment 57 provides the method of any one of Embodiments 37-56, wherein the composition is a syrup.

Embodiment 58 provides the appetite suppressing composition of any one of Embodiments 37-57, wherein the composition is included in a food product.

Embodiment 59 provides the appetite suppressing composition of Embodiment 58, wherein the food product is chosen from one or more of a dairy product, a yoghurt, an ice cream, a milk-based drink, a milk-based garnish, a pudding, a milkshake, an egg custard, a cheese, a nutrition bar, an energy bar, a breakfast bar, a confectionery, a bakery product, a cracker, a cookie, a biscuit, a cereal chip, a snack product, an ice tea, a fruit juice, a diet drink, a soda, a sports drink, a powdered drink mixture for dietary supplementation, an infant and baby food, a calcium-supplemented orange juice, a bread, a croissants, a breakfast cereal, a pasta, a noodle, a spread, a sugar-free biscuit, a sugar free chocolate, a calcium chew, a meat product, a mayonnaise, a salad dressing, a nut butter, a sauce, a syrup, a crisp, a puff, a candy, and a soup.

Embodiment 60 provides the method of Embodiment 58, wherein the syrup is a first food product that at least partially coats an external surface of a second food product.

Embodiment 61 provides the appetite suppressing composition of any one of Embodiments 58 or 60, wherein the syrup is a first food product and is at least partially dispersed within a second food product.

Embodiment 62 provides the appetite suppressing composition of any one of Embodiments 37-61, wherein and the chicory flour is located a food product and the amount of the cannabinoid is dissolved in a syrup comprising an oligofructose.

Embodiment 63 provides the appetite suppressing composition of Embodiment 62, wherein the syrup comprising the oligofructose is applied to an external surface of the food.

Embodiment 64 provides the appetite suppressing composition of Embodiment 62, wherein the oligofructose is inulin.

Embodiment 65 provides the appetite suppressing composition of any one of Embodiments 37-64, wherein the composition is included in an animal feed product.

Embodiment 66 provides the appetite suppressing composition of any one of Embodiments 37-65, wherein the chicory flour comprises about 50% (w/w) to about 90% (w/w) oligofructose.

Embodiment 67 provides the appetite suppressing composition of any one of Embodiments 37-66, wherein the chicory flour comprises a percentage (w/w) of oligofructose that is equal to or greater than a percentage (w/w) of oligofructose found in taproots of Cichorium intybus.

Embodiment 68 provides the appetite suppressing composition of any one of Embodiments 37-68, wherein the amount of the cannabinoid in the composition is less than the minimum amount of cannabinoid required to suppress appetite in a subject in the absence of the chicory flour.

Embodiment 69 provides the appetite suppressing composition of any one of Embodiments 37-68, wherein the amount of chicory flour in the composition is less than a minimum amount of chicory flour required to suppress appetite in a subject in the absence of the cannabinoid.

Embodiment 70 provides an appetite suppression composition comprising:
(A) an amount of a cannabinoid according to Formula IIA or Formula IIB:

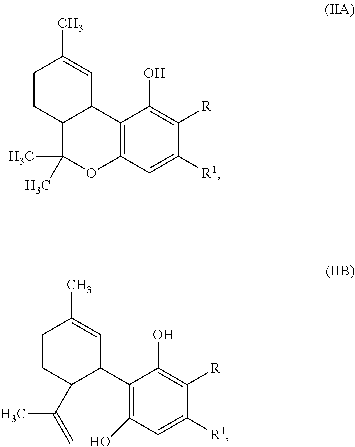

that is produced by a process comprising:
(i) contacting a compound according to Formula I:

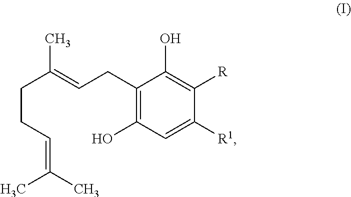

with a cannabinoid synthase in the presence of a solvent to produce the compound according to Formula II
wherein:
R is —H, COOH, or —(CH₂)ₙCOOH;
R¹ (C₁-C₁₀) alkyl; and
n is 1, 2, 3, 4, 5, or 6.
(B) an amount of chicory flour comprising about 10% (w/w) to about 90% (w/w) less 11β,13-dihydrolactucin than a chicory flour made from the taproots of Cichorium intybus.

Embodiment 71 provides the appetite suppression composition of Embodiment 70, wherein the cannabinoid synthase tetrahydrocannabinolic acid (THCA) synthase tetrahydrocannabivarin acid (THCVA) synthase, or cannabidolic acid (CBDA) synthase.

Embodiment 72 provides the appetite suppression composition of Embodiment 70, wherein R is —COOH and R¹ is propyl.

Embodiment 73 provides the appetite suppression composition of Embodiment 70, wherein the compound according to Formula IIA is tetrahydrocannabivarin carboxylic acid (THCVA).

Embodiment 74 provides the appetite suppression composition of Embodiment 73, further comprising:
(ii) decarboxylating THCVA to produce THC-v.

Embodiment 75 provides the appetite suppressing composition of any one of Embodiments 70-74, wherein the chicory flour is produced by a process comprising:
(iii) cooking chicory taproots at a temperature ranging from about 40° C. to about 100° C.;
(iv) drying the chicory taproots to produce chicory chips; and
(v) milling the chicory chips to produce the chicory flour.

Embodiment 76 provides the appetite suppressing composition of Embodiment 75, wherein cooking the chicory taproots comprises one or more of boiling, microwaving, steaming, and blanching unroasted chicory taproots.

Embodiment 77 provides the appetite suppressing composition of Embodiment 76, wherein the chicory taproots are blanched at a temperature ranging from about 60° C. to about 70° C.

Embodiment 78 provides the appetite suppressing composition of Embodiment 75, wherein the chicory root chips are dried in a furnace at a temperature ranging from about 145° C. to about 180° C. or in a fluidized bed dryer.

Embodiment 79 provides the appetite suppressing composition of any one of Embodiments 70-78, wherein the composition comprises about 0.01% (w/w) to about 20% (w/w) cannabinoid.

Embodiment 80 provides the appetite suppressing composition of any one of Embodiments 70-79, wherein the composition comprises about 1% (w/w) to about 60% (w/w) chicory flour.

Embodiment 81 provides the appetite suppressing composition of any one of Embodiments 70-80, wherein, the chicory flour comprises less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 10% (w/w) of a protein.

Embodiment 82 provides the appetite suppressing composition of any one of Embodiments 70-81, wherein the chicory flour includes substantially equivalent levels of 8-deoxylactucin and lactucopicrin compared to a chicory flour made from the taproots of Cichorium intybus.

Embodiment 83 provides the appetite suppressing composition of any one of Embodiments 70-82, wherein the composition is a syrup.

Embodiment 84 provides the appetite suppressing composition of any one of Embodiments 70-83, wherein the composition is included in a food product.

Embodiment 85 provides the appetite suppressing composition of Embodiment 84, wherein the food product is chosen from one or more of a dairy product, a yoghurt, an ice cream, a milk-based drink, a milk-based garnish, a pudding, a milkshake, an egg custard, a cheese, a nutrition bar, an energy bar, a breakfast bar, a confectionery, a bakery product, a cracker, a cookie, a biscuit, a cereal chip, a snack product, an ice tea, a fruit juice, a diet drink, a soda, a sports drink, a powdered drink mixture for dietary supplementation, an infant and baby food, a calcium-supplemented orange juice, a bread, a croissants, a breakfast cereal, a pasta, a noodle, a spread, a sugar-free biscuit, a sugar free chocolate, a calcium chew, a meat product, a mayonnaise, a salad dressing, a nut butter, a sauce, a syrup, a crisp, a puff, a candy, and a soup.

Embodiment 86 provides the appetite suppressing composition of Embodiment 84, wherein the syrup is a first food product that at least partially coats an external surface of a second food product.

Embodiment 87 provides the appetite suppressing composition of 84, wherein the syrup is a first food product and is at least partially dispersed within a second food product.

Embodiment 88 provides the method of any one of Embodiments 70-87, wherein and the chicory flour is located a food product and the amount of the cannabinoid is dissolved in a syrup comprising an oligofructose.

Embodiment 89 provides the appetite suppressing composition of Embodiment 88, wherein the syrup comprising the oligofructose is applied to an external surface of the food.

Embodiment 90 provides the appetite suppressing composition of 88, wherein the oligofructose is inulin.

Embodiment 91 provides the appetite suppressing composition of any one of Embodiments 70-90, wherein the composition is included in an animal feed product.

Embodiment 92 provides the appetite suppressing composition of any one of Embodiments 70-91, wherein the chicory flour comprises about 50% (w/w) to about 90% (w/w) oligofructose.

Embodiment 93 provides the appetite suppressing composition of any one of Embodiments 70-92, wherein the chicory flour comprises a percentage (w/w) of oligofructose that is equal to or greater than a percentage (w/w) of oligofructose found in taproots of Cichorium intybus.

Embodiment 94 provides the appetite suppressing composition of any one of Embodiments 70-93, wherein the amount of THC-v in the composition is less than the minimum amount of THC-v required to suppress appetite in a subject in the absence of the chicory flour.

Embodiment 95 provides the appetite suppressing composition of any one of Embodiments 70-94, wherein the amount of chicory flour in the composition is less than the minimum amount of chicory flour required to suppress appetite in a subject in the absence of the THC-v.

Embodiment 96 provides the appetite suppressing composition of any one of Embodiments 70-95, wherein the compound according to Formula 1 is located within a bioreactor containing the solvent and the cannabinoid synthase.

Embodiment 97 provides the appetite suppressing composition of Embodiment 96, wherein the bioreactor comprises:
a control mechanism configured to control at least one condition of the bioreactor.

Embodiment 98 provides the appetite suppressing composition of Embodiment 97, wherein the condition of the bioreactor is selected from temperature, solvent, pressure and, pH, and combinations thereof.

Embodiment 99 provides the appetite suppressing composition of Embodiment 74, wherein decarboxylating THCVA includes contacting the THCVA with a weak base.

Embodiment 100 provides the appetite suppressing composition of Embodiment 99, wherein the weak base is sodium carbonate.

Embodiment 101 provides a composition comprising:
(A) an amount of a cannabinoid; and
(B) an amount of chicory flour comprising about 10% (w/w) to about 90% (w/w) less 11β,13-dihydrolactucin than a chicory flour made from the taproots of Cichorium intybus.

Embodiment 102 provides the composition of Embodiment 101, wherein the cannabinoid is a compound according to Formula IIA or Formula IIB:

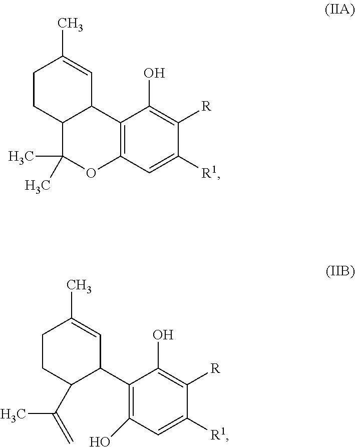

and wherein the compound of Formula IIA is formed from a method comprising:
(i) contacting a compound according to Formula I:

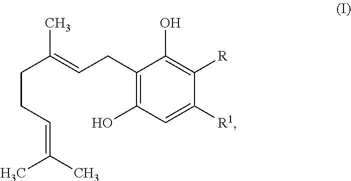

with a cannabinoid synthase in the presence of a solvent to produce the compound according to Formula II
wherein:
R is —H, COOH, or —(CH₂)ₙCOOH;
R¹ is (C₁-C₁₀) alkyl; and
n is 1, 2, 3, 4, 5, or 6.

Embodiment 103 provides the composition of Embodiment 102, wherein the cannabinoid synthase is tetrahydrocannabinolic acid (THCA) synthase tetrahydrocannabivarin acid (THCVA) synthase, or cannabidolic acid (CBDA) synthase.

Embodiment 104 provides the composition of Embodiment 102, wherein R is —COOH and R¹ is propyl.

Embodiment 105 provides the composition of Embodiment 104, wherein the compound according to Formula IIA is tetrahydrocannabivarin carboxylic acid (THCVA).

Embodiment 106 provides the composition of Embodiment 102, further comprising:
(ii) decarboxylating THCVA to produce THC-v.

Embodiment 107 provides the composition of Embodiment 102, further comprising:
(ii′) oxidizing the Formula IIA compound to produce a compound according to Formula III:

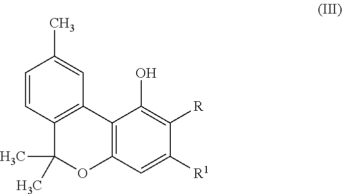

Embodiment 108 provides the composition of Embodiment 102, wherein R is —COOH and R¹ is propyl or pentyl.

Embodiment 109 provides the composition of Embodiment 102, further comprising the step of decarboxylation by heating a solution of the Formula IIA compound or by exposing a solution of the Formula IIA compound to UV-light.

Embodiment 110 provides the composition of Embodiment 109, wherein R¹ is pentyl, and the compound according to Formula III is Cannabinol (CBN).

Embodiment 111 provides the composition of Embodiment 109, wherein R¹ is propyl, and the compound according to Formula III is Cannabivarin (CBV).

Embodiment 112 provides the composition of Embodiment 101, wherein the cannabinoid is selected from the group consisting of Cannabivarin (CBV), Cannabinol (CBN), Cannabigerol (CBG). Cannabigerolic acid (CBGA), Cannabigerovarin (CBGV), Cannabigerovarinic acid (CBGVA), Cannabidiol (CBD), Cannabidiolic acid (CBDA), Cannabidivarin (CBDV), Cannabidivarinic acid (CBDVA), Cannabichromene (CBC), Cannabichromenic acid (CBCA), Cannabichromevarin (CBCV), Cannabichromevarinic acid (CBCVA), Δ9-Tetrahydrocannabinol (THC), Δ9-Tetrahydrocannabinolic acid (Δ9-THCA), Δ9-Tetrahydrocannabivarin (THCV), Δ9 Tetrahydrocannabivarinic acid (THCVA), Cannabinol (CBN), Cannabinolic acid (CBNA), Cannabivarin (CBNVA), Cannabielsoin (CBE), Cannabicyclol (CBL), Cannabicyclolic Acid (CBLA), Tetrahydrocannabivarin (THV), a prodrug of any cannabinoid thereof, and any combination thereof.

Embodiment 113 provides the composition of any one of Embodiments 101-112, wherein the chicory flour is produced by a process comprising:
(iii) cooking chicory taproots at a temperature ranging from about 40° C. to about 100° C.;
(iv) drying the chicory taproots to produce chicory chips; and
(v) milling the chicory chips to produce the chicory flour.

Embodiment 114 provides the composition of Embodiment 113, wherein cooking the chicory taproots comprises one or more of boiling, microwaving, steaming, and blanching unroasted chicory taproots.

Embodiment 115 provides the composition of Embodiment 114, wherein the chicory taproots are blanched at a temperature ranging from about 60° C. to about 70° C.

Embodiment 116 provides the composition of Embodiment 113, wherein the chicory root chips are dried in a furnace at a temperature ranging from about 145° C. to about 180° C., or in a fluidized bed dryer.

Embodiment 117 provides the composition of any one of Embodiments 101-116, wherein the composition comprises about 0.01% (w/w) to about 20% (w/w) the cannabinoid. Embodiment 118 provides the composition of any one of Embodiments 101-117, wherein the composition comprises about 1% (w/w) to about 60% (w/w) chicory flour.

Embodiment 119 provides the composition of any one of Embodiments 101-118, wherein, the chicory flour comprises less than 1% (w/w) of a sugar, less than 5% (w/w) of a fat, about 10% (w/w) to about 20% (w/w) of an insoluble fiber, about 50% (w/w) to about 75% (w/w) of a soluble fiber, and about 2.5% (w/w) to about 10% (w/w) of a protein.

Embodiment 120 provides the composition of any one of Embodiments 101-119, wherein the chicory flour includes substantially equivalent amounts (w/w) of 8-deoxylactucin and lactucopicrin compared to a chicory flour made from the taproots of Cichorium intybus.

Embodiment 121 provides the composition of any one of Embodiments 101-120, wherein the composition is a syrup.

Embodiment 122 provides the composition of any one of Embodiments 101-121, wherein the composition is included in a food product.

Embodiment 123 provides the composition of Embodiment 122, wherein the food product is chosen from one or more of a dairy product, a yoghurt, an ice cream, a milk-based drink, a milk-based garnish, a pudding, a milkshake, an egg custard, a cheese, a nutrition bar, an energy bar, a breakfast bar, a confectionery, a bakery product, a cracker, a cookie, a biscuit, a cereal chip, a snack product, an ice tea, a fruit juice, a diet drink, a soda, a sports drink, a powdered drink mixture for dietary supplementation, an infant and baby food, a calcium-supplemented orange juice, a bread, a croissants, a breakfast cereal, a pasta, a noodle, a spread, a sugar-free biscuit, a sugar free chocolate, a calcium chew, a meat product, a mayonnaise, a salad dressing, a nut butter, a sauce, a syrup, a crisp, a puff, a candy, and a soup.

Embodiment 124 provides the composition of Embodiment 123, wherein the syrup is a first food product that at least partially coats an external surface of a second food product.

Embodiment 125 provides the composition of Embodiment 123, wherein the syrup is a first food product and is at least partially dispersed within a second food product.

Embodiment 126 provides the composition of any one of Embodiments 101-125, wherein the chicory flour is located a food product and the amount of the cannabinoid is dissolved in a syrup comprising an oligofructose.

Embodiment 127 provides the composition of Embodiment 126, wherein the syrup comprising the oligofructose is applied to an external surface of the food.

Embodiment 128 provides the composition of Embodiment 126, wherein the oligofructose is inulin.

Embodiment 129 provides the composition of any one of Embodiments 101-128, wherein the composition is included in an animal feed product.

Embodiment 130 provides the composition of any one of Embodiments 101-129, wherein the chicory flour comprises about 50% (w/w) to about 90% (w/w) oligofructose.

Embodiment 131 provides the composition of any one of Embodiments 101-130, wherein the chicory flour comprises a percentage (w/w) of oligofructose that is equal to or greater than a percentage (w/w) of oligofructose found in taproots of Cichorium intybus.

1. A method of making an appetite suppressing composition comprising (i) combining:
(A) an amount of a cannabinoid; and
(B) an amount of chicory flour comprising about 10% (w/w) to about 90% (w/w) less 11β,13-dihydrolactucin than a chicory flour made from the taproots of Cichorium intybus.

2. The method of claim 1, wherein the cannabinoid is a formula according to Formula IIA or Formula IIB: 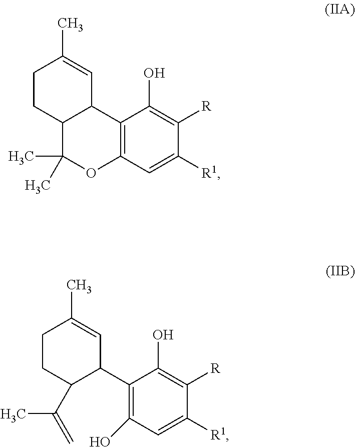
and wherein the method further comprises:
(ii) contacting a compound according to Formula I: 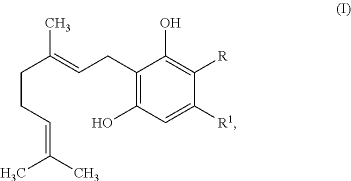
with a cannabinoid synthase in the presence of a solvent to produce the compound according to Formula II
wherein:
R is —H, COOH, or —(CH₂)ₙCOOH;
R¹ (C₁-C₁₀) alkyl; and
n is 1, 2, 3, 4, 5, or 6.

3. The method of claim 2, wherein the cannabinoid synthase is tetrahydrocannabinolic acid (THCA) synthase tetrahydrocannabivarin acid (THCVA) synthase, or cannabidolic acid (CBDA) synthase.

4. (canceled)

5. (canceled)

6. The method of claim 1, wherein the compound according to Formula IIA is tetrahydrocannabivarin carboxylic acid (THCVA), further comprising:
(iii) decarboxylating THCVA to produce THC-v.

7.-11. (canceled)

12. The method of claim 1, wherein the cannabinoid is selected from the group consisting of Cannabivarin (CBV), Cannabinol (CBN), Cannabigerol (CBG), Cannabigerolic acid (CBGA), Cannabigerovarin (CBGV), Cannabigerovarinic acid (CBGVA), Cannabidiol (CBD), Cannabidiolic acid (CBDA), Cannabidivarin (CBDV), Cannabidivarinic acid (CBDVA), Cannabichromene (CBC), Cannabichromenic acid (CBCA), Cannabichromevarin (CBCV), Cannabichromevarinic acid (CBCVA), Δ9-Tetrahydrocannabinol (THC), Δ9-Tetrahydrocannabinolic acid (Δ9-THCA), Δ9-Tetrahydrocannabivarin (THCV), Δ9 Tetrahydrocannabivarinic acid (THCVA), Cannabinol (CBN), Cannabinolic acid (CBNA), Cannabivarin (CBNVA), Cannabielsoin (CBE), Cannabicyclol (CBL), Cannabicyclolic Acid (CBLA), Tetrahydrocannabivarin (THV), a prodrug of any cannabinoid thereof, and any combination thereof.

13. The method of claim 1, further comprising:
(iv) cooking chicory taproots at a temperature ranging from about 40° C. to about 100° C.;
(v) drying the chicory taproots to produce chicory chips; and
(vi) milling the chicory chips to produce the chicory flour.

14.-16. (canceled)

17. The method of claim 1, wherein the composition comprises at least one of about 0.01% (w/w) to about 20% (w/w) cannabinoid and about 1% (w/w) to about 60% (w/w) chicory flour.

18.-36. (canceled)

37. An appetite suppressing composition comprising:
(A) an amount of a cannabinoid; and
(B) an amount of chicory flour comprising about 10% (w/w) to about 900% (w/w) less 11β,13-dihydrolactucin than a chicory flour made from the taproots of Cichorium intybus;
wherein an amount of composition suppresses the appetite of a subject who is administered the composition to a greater extent than of a subject who is administered the same amount of the cannabinoid or chicory flour.

38.-47. (canceled)

48. The appetite suppressing composition of claim 37, wherein the cannabinoid is selected from the group consisting of Cannabivarin (CBV), Cannabinol (CBN), Cannabigerol (CBG), Cannabigerolic acid (CBGA), Cannabigerovarin (CBGV), Cannabigerovarinic acid (CBGVA), Cannabidiol (CBD), Cannabidiolic acid (CBDA), Cannabidivarin (CBDV), Cannabidivarinic acid (CBDVA), Cannabichromene (CBC), Cannabichromenic acid (CBCA), Cannabichromevarin (CBCV), Cannabichromevarinic acid (CBCVA), Δ9-Tetrahydrocannabinol (THC), Δ9-Tetrahydrocannabinolic acid (THCA), Δ9-Tetrahydrocannabivarin (THCV), Δ9 Tetrahydrocannabivarinic acid (THCVA), Cannabinol (CBN), Cannabinolic acid (CBNA), Cannabivarin (CBNVA), Cannabielsoin (CBE), Cannabicyclol (CBL), Cannabicyclolic Acid (CBLA), Tetrahydrocannabivarin (THV), a prodrug of any cannabinoid thereof, and any combination thereof.

49.-52. (canceled)

53. The appetite suppressing composition of claim 37, wherein the composition comprises at least one of about 0.01% (w/w) to about 20% (w/w) the cannabinoid and about 1% (w/w) to about 60% (w/w) chicory flour.

54.-56. (canceled)

57. The method of claim 37, wherein the composition further comprises a food product.

58. The appetite suppressing composition of claim 37, wherein the composition is a syrup at least partially coating the food product.

59.-67. (canceled)

68. The appetite suppressing composition of claim 37, wherein at least one of the amount of the cannabinoid in the composition is less than the minimum amount of cannabinoid required to suppress appetite in a subject in the absence of the chicory flour and the amount of chicory flour in the composition is less than a minimum amount of chicory flour required to suppress appetite in a subject in the absence of the cannabinoid.

69. (canceled)

70. An appetite suppression composition comprising:
(A) an amount of a cannabinoid according to Formula IIA or Formula IIB: 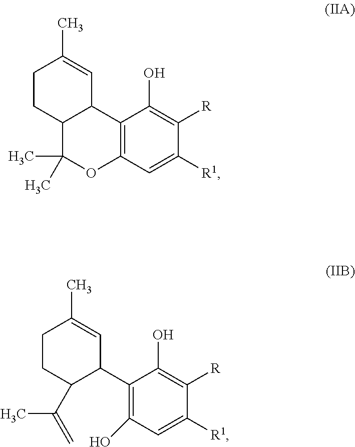
that is produced by a process comprising:
(i) contacting a compound according to Formula I: 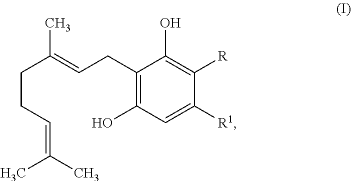
with a cannabinoid synthase in the presence of a solvent to produce the compound according to Formula II
wherein:
R¹ is —H, COOH, or —(CH₂)ₙCOOH;
R¹ (C₁-C₁₀) alkyl; and
n is 1, 2, 3, 4, 5, or 6,
(B) an amount of chicory flour comprising about 10% (w/w) to about 90% (w/w) less 11β,13-dihydrolactucin than a chicory flour made from the taproots of Cichorium intybus.

71. The appetite suppression composition of claim 70, wherein the cannabinoid synthase tetrahydrocannabinolic acid (THCA) synthase tetrahydrocannabivarin acid (THCVA) synthase, or cannabidolic acid (CBDA) synthase.

72. The appetite suppression composition of claim 70, wherein R is —COOH and R¹ is propyl.

73. The appetite suppression composition of claim 70, wherein the compound according to Formula IIA is tetrahydrocannabivarin carboxylic acid (THCVA).

74. The appetite suppression composition of claim 73, further comprising:
(ii) decarboxylating THCVA to produce THC-v.

75. The appetite suppressing composition of claim 70, wherein the chicory flour is produced by a process comprising:
(iii) cooking chicory taproots at a temperature ranging from about 40° C. to about 100° C.;
(iv) drying the chicory taproots to produce chicory chips; and
(v) milling the chicory chips to produce the chicory flour.

76.-82. (canceled)

83. The appetite suppressing composition of claim 70, wherein the composition further includes a food product.

84.-131. (canceled)

132. The appetite suppressing composition of claim 83, wherein the composition is a syrup at least partially coating the food product.
